(19) United States
(12) Patent Application Publication
(10) Pub. No.: US 2007/0208395 A1
(43) Pub. Date: Sep. 6, 2007

(54) Phototherapy Device and Method of Providing Phototherapy to a Body Surface

(76) Inventors: Norbert H. Leclerc, Mountain View, CA (US); Brendan Jude Moran, Redwood City, CA (US); James R. Flom, San Mateo, CA (US); Michael Gertner, Menlo Park, CA (US); Jonathan L. Podmore, San Carlos, CA (US); Michael Rode, Sunnyvale, CA (US); John E. Crowe, Menlo Park, CA (US); Erica Rogers, Redwood City, CA (US)

Correspondence Address: WILSON SONSINI GOODRICH & ROSATI, 650 PAGE MILL ROAD, PALO ALTO, CA, 94304-1050, US

(21) Appl. No.: 11/686,121

(22) Filed: Mar. 14, 2007

Related U.S. Application Data
(63) Continuation-in-part of application No. 11/276,787, filed on Mar. 14, 2006.
Application No. 11/276,787 is a continuation-in-part of application No. 11/244,812, filed on Oct. 5, 2005.
(60) Provisional application No. 60/882,439, filed on Dec. 28, 2006.

(51) Int. Cl. A61N 5/06

(52) U.S. Cl. 607/86

(57) ABSTRACT

A method and apparatus is described for treating a target body surface using a radiation applicator. The therapeutic treatment apparatus adapted to conform to a patients body. The treatment apparatus comprises a plurality of light sources coupled with a flexible substrate, a light integrator in at least a portion of the optical path between the light source and the patient's body surface, a power supply, and a controller.

### CROSS-REFERENCE

This application is a continuation-in-part application of Ser. No. 11/276,787 filed Mar. 14, 2006, which in turn is a continuation-in-part application of Ser. No. 11/244,812, filed Oct. 5, 2005, which are incorporated herein by reference in their entirety and to which application priority is claimed under 35 U.S.C. §120. This application also claims benefit of U.S. Provisional Application No. 60/882,439 filed Dec. 28, 2006, which is also incorporated herein by reference in its entirety.

### BACKGROUND OF THE INVENTION

1. Field of the Invention

The invention relates to devices and methods of use for small, portable devices adapted and configured to deliver phototherapeutic treatments to a select area of a skin surface to treat medical skin disorders or to perform cosmetic dermatological therapies. More specifically, the devices comprise a plurality of light sources and are flexible such that the devices are adapted to conform to a body surface while providing controlled light distribution.

2. Background of the Invention

The therapeutic use of light has been shown to be effective in the treatment of various medical conditions. For example, ultraviolet (“UV”) light has been used for medical applications, such as the treatment of psoriasis, atopic dermatitis and vitiligo. Ultraviolet lasers and lamps with UVB light are currently used for treating these conditions. In some cases, PUVA, combining UVA light with psoralen, is used to treat psoriasis. Treatment typically requires a patient visiting their physician approximately 3 visits per week for up to 16 weeks. Photodynamic therapy (PDT) is a recently evolving modality for the treatment of skin conditions such as actinic keratosis, acne, and skin cancer. This treatment involves the use of light, including red light, with a photosensitizer. The photosensitizer is either administered orally or topically.

It has been shown that UVC light in approximately 254 nm wavelength can sterilize microorganisms including, but not limited to, viruses and bacteria. Skin infections, then, can be treated with UVC light. Some infections, including Staph. Aureus, are resistant to most antibiotics. Even these resistant microorganisms can be sterilized with UVC light. However, there are no small, portable devices currently available to deliver this type of treatment.

Phototherapy is also used for certain cosmetic dermatological conditions. Procedures to remove unwanted hair, remove vascular lesions or pigmentation, eliminate acne, and rejuvenate the skin, for example, are becoming common. These treatments typically use light in either visible wavelengths (400-800 nm) or near infrared and infrared wavelengths (800-2000 nm). Common devices for these treatments are lasers; however, other light sources, including LED's, are also available for certain of these treatments like acne. These treatments also typically require a series of visits to a physician's office. Skin tanning is another cosmetic procedure using light, typically UVA. Tanning beds with bulbs which can illuminate a large body surface area are commonly used for this purpose.

Phototherapy has proven to be a viable and desirable treatment strategy for the above mentioned skin ailments and cosmetic procedures. However, current phototherapy treatment available to patients has several shortcomings. In one treatment strategy for psoriasis, patients must visit a physician office and sit unclothed in a phototherapy chamber for a period of time. In this type of treatment, areas of healthy skin are also exposed to the treatment dose which may cumulatively lead to damage to skin that was originally normal and healthy. Additionally, this treatment requires numerous visits to a physician office to receive a course of therapy. The expense and loss of productivity due to these visits is a compelling reason for the advent of a new technology. Additionally, lasers and bulb light sources are undesirably large. In a clinic, they reduce available space for other medical equipment. Additionally, these light sources can be prohibitively expensive.

An alternative therapeutic device that is small, portable, and meets or exceeds the therapeutic benefit provided by the above devices is hereby described. In order to provide therapeutic benefit, the light distribution of the device must be selectively controlled. Additionally, a suitable device or mechanism to treat only the target region of skin is desirable.

A variety of devices are known for delivering light and/or radiation. For example, PCT Publication WO 2005/000389 to Fiset for Skin Tanning and Light Therapy Incorporating Light Emitting Diodes (see also, U.S. Patent Pub. 2004/0232339 to Lanoue for Hyperspectral Imaging Workstation Having Visible/Near-Infrared and Ultraviolet Image Sensors). U.S. Pat. No. 6,290,713 to Russell for Flexible Illuminators for Phototherapy; U.S. Patent Pub. 2004/0176824 to Weckworth for Method and Apparatus for the Repigmentation of Human Skin; U.S. Pat. No. 6,730,113 to Eckhardt et al. for Method and Apparatus for Sterilizing or Disinfecting A Region Through a Bandage; U.S. Pat. No. 6,096,066 to Chen et al. for Conformal Patch for Administering Light Therapy to Subcutaneous Tumors; and U.S. Pat. No. 6,645,230 to Whitehurst for Therapeutic Light Source and Method. A variety of devices are also known for providing bandages or dressing, including, for example, U.S. Pat. No. 2,992,644 to Plantinga et al. for Dressing; U.S. Pat. No. 3,416,525 to Yeremian for Stabilized Non-Adherent Dressing; U.S. Pat. No. 3,927,669 to Glatt for Bandage Construction; U.S. Pat. No. 4,126,130 to Cowden for Wound Protective Device; U.S. Pat. No. 4,561,435 to McKnight et al. for Wound Dressing; U.S. Pat. No. 4,616,644 to Saferstein et al. for Hemostatic Adhesive Bandage; U.S. Pat. No. 4,671,266 to Lengyel et al. for Blister Bandage; U.S. Pat. No. 4,901,714 to Jensen for Bandage; U.S. Pat. No. 5,336,209 to Porilli for Multi-Function Wound Protection Bandage and Medicant Delivery System with Simultaneous Variable Oxygenation; U.S. Pat. No. 5,954,679 to Baranitsky for Adhesive Bandage; 6,096,066 to Chen for Conformal Patch for Administering Light Therapy to Subcutaneous Tumors; U.S. Pat. No. 6,343,604 B1 to Beall for Protective Non Occlusive Wound Shield; U.S. Pat. No. 6,384,294 B1 to Levin for Protective Bandages Including Force-Transmission-Impeding Members Thereof; U.S. Pat. No. 6,443,978 to Zharov for Photomatrix Device; U.S. Pat. No. 5,616,140 to Prescott for Method and Apparatus for Therapeutic Laser Treatment; U.S. Pat. No. 5,913,883 to Alexander et al for Therapeutic Facial Mask; U.S. Pat. No. 6,866,678 to Shenderova et al. for Phototherapeutic Treatment Methods and Apparatus; U.S. Pat. No. 6,986,782 to Chen et al. for Ambulatory Photodynamic Therapy; U.S. Pat. No. 6,955,684 to Savage Jr., et al., for Portable Light Delivery Apparatus and Methods; and U.S. Patent Publications US 2001/0028943 A1 to Mashiko et al. for Adhesive Film for Adhesive Bandage Using Said Adhesive Film; US 2002/0128580 A1 to Carlson for Self-Adhering Friction Reducing Liner and Method of Use; US 2002/0183813 A1 to Augustine et al. for Treatment Apparatus with a Heater Adhesively Joined to the Bandage; US 2003/0199800 A1 to Levin for Bandage Including Perforated Gel; US 2003/0163074 A1 to McGowan et al. for Wound Dressing Impervious to Chemical and Biological Agents; US 2003/0143264 A1 to Margiotta for Topical Anesthetic-Antiseptic Patch; US 2004/0087884 A1 to Haddock et al. for Textured Breathable Films and Their Use as Backing Material for Bandages; US 2004/0049144 A1 to Cea for Hypoallergenic Bandage; US 2004/0260365 to Groseth et al. for Photodynamic Therapy Lamp; and US 2005/0010154 A1 to Wright et al. for Adhesive Bandage for Protection of Skin Surface.

### SUMMARY OF THE INVENTION

The invention relates to a photodynamic or radiation treatment apparatus having a light and/or radiation source adapted to irradiate a target portion of a body.

Provided is a device to deliver phototherapy and photodynamic therapy in a spatially uniform dose to an area of a body surface in need. The phototherapy treatment includes ultraviolet, visible, and infrared light as is necessitated by the specific condition to be treated. This device is specifically designed and constructed to conform to an arbitrary body surface to optimize the therapeutic options for a patient. For example, an embodiment of the described device has configurable flexibility to provide phototherapy to the face, back, knee, and elbow in separate instances without substantially changing in form or general functionality.

In accordance with the invention, therapeutic light is generated by small, lightweight light sources such as LEDs or lasers and delivered via a flexible, conformal optically transmissive element to a body surface. It is intended that this element make direct, intimate contact with a body surface. The element is both thin in profile and made at least in part from a soft, flexible material thus engendering its conformal nature.

Still further in accordance with the described invention, the device is intended to be securely attached to a patient via an adhesive, a strap, or other mechanism such that the recipient of the therapy is minimally encumbered during treatment. Additionally, light sources are controlled by a small microprocessor and powered by a battery. The combination of the preceding two qualities enables the patient to, for example, be free to move about during treatment.

Still further in accordance with the described invention, the light delivery element is in part composed of rigid or semi-rigid optical integrator elements that are in intimate contact with the body surface and adhered to a flexible substrate. One or more light sources are associated with each of these optical integrator elements. The optical transmission properties of each integrator element are such that a uniform light distribution is transmitted to the body surface in which it is in contact. The spacing and configuration geometry of the light integrator elements essentially determine the total body surface area receiving treatment. Therefore, the ensemble effect of such elements on a flexible substrate is to substantially conform to a body surface as well as deliver a uniform therapeutic treatment over the same surface.

Still further in accordance with the described invention is an intermediary targeting mask to be used in concert with the phototherapy delivery element. This targeting mask is used in regions where an affected area is irregular in shape and overall smaller in size as compared to the therapy device. Its function is to be placed in between the device and the body surface and selectively expose affected surface areas to the phototherapy treatment while simultaneously minimizing or eliminating such a treatment light from reaching unaffected neighboring regions of healthy skin surface. Still further in accordance with the described invention is the ability to detect the zone for treatment and subsequently power a subset of the light sources on the phototherapy delivery element.

Further aspects, details, and embodiments of the present invention will be understood by those of skill in the art upon reading the following detailed description of the invention and the accompanying drawings.

An aspect of the invention is directed to a therapeutic treatment apparatus adapted and configured to conform to a target region of a patient. An apparatus according to this embodiment includes, a plurality of light sources adapted and configured to couple to a flexible substrate to deliver light to the target region, a power supply coupled to the light sources and operable to provide power to the light sources, and a controller coupled to the light sources and the power supply and operable to control the operation of the light sources, wherein the therapeutic treatment apparatus is disposed adjacent a light integrator in at least a portion of an optical path for the light between the light sources and the target region of the patient during deployment.

The apparatus or devices of the invention can further be adapted such that each light source further comprises one or more light emitting diodes or one or more laser diodes. Diodes can be positioned relative to a surface of the flexible substrate to deliver light at one or more prescribed angles with respect to the target region of the patient's body surface. A variety of wavelengths are suitable for the invention, including, for example, wavelengths in the range of 200-2000 nm. Flexible substrates can be formed from any suitable material that achieves the conformable aspect, including, for example, rubber, cloth; thermoplastic elastomer, thermoplastic, fabric, or flexible metal. Furthermore, the devices can further include a single-use layer positioned between light delivered by the light sources and the target region of the patient's body surface. Additionally, the light integrator can be formed from a rigid or semi-rigid material further adapted and configured to at least partially transmit light. The light integrator facilitates and integrates the transmission of light to the target area. For example, the light integrator can be adapted and configured to internally reflect the light to substantially uniformly distribute the light onto the target region of the patient's body surface or adapted and configured to use a total internal reflection to distribute the light onto the target region of the patient's body surface, such as where the internal reflection is substantially uniform. Additionally, one or more lower edges of the light integrator can further be adapted and configured to have a minimum radius of curvature of 0.5 mm and maximum radius of curvature of 25 cm. In some embodiments, it may be desirable to form the light integrator from silicone rubber. The light integrator in some embodiments, is at least partially further comprised of a support structure adapted and configured to separate the light sources and the target region of the patient's body surface. A suitable support structure can further be partially reflective and/or be adapted and configured to contact <15% of the target region of the patient's body surface.

Light integrators used with the therapeutic treatment apparatus can further comprise a lens adapted and configured to be positioned between the light source and the target region of the patient's body surface. Additionally, the substrates can further comprises a substrate at least partially transmissive to light, such as silicone rubber. A variety of controllers are suitable for use with the invention. The controllers can use a shared power source as the light sources, or an independent power source. The controllers can further be configurable to selectively control one or more treatment parameters, such as for a specific region of the patient, and/or to provide one or more patient specific codes. Treatment parameters can include, for example, duration of treatment, treatment frequency, or total numbers of available treatments.

A variety of sensors can be provided in conjunction with the apparatus. The sensors can be configured to detect, for example, proper placement of the apparatus on patient.

Depending upon the target region to be treated, the apparatus may further be configured to provide an attachment mechanism in order to faclitate deployment of the device onto the patient's target region. The attachment mechanism can include, for example, the use of adhesives or adhesive sections, straps, material or fabric wraps, or a cuff.

An additional feature of the apparatus can include a heat collector adapted and configured to absorb heat generated by the light sources. The heat collector can further comprise, for example, a material, such as a heat absorbing material or a heat conductive material, integrated with each light source. Integrating a heat absorber or heat conducter facilitates drawing at least some of the heat away from the surface of the skin.

In still another embodiment of the invention, a targeting mask adapted and configured to at least partially block therapeutic light from a first region of a patient (e.g., healthy skin that does not require treatment) and at least partially transmit therapeutic light to a second region of a patient (e.g., skin having a lesion to be treated) is provided. The targeting mask can be configured to integrate with the apparatus or can further comprise its own an attachment mechanism, such as adhesive, adapted and configured to attach the targeting mask to the patient. Typically, the mask will be comprised of at least one flexible material, such as foam, rubber, plastic, synthetic fabric, natural fabric, or elastomer, to facilitate placement on a patient.

In another aspect of the invention, a therapeutic treatment apparatus is provided that is adapted and configured to contact a target surface of a patient. The apparatus comprising: a light source, a power supply coupled to the light source and operable to provide power to the light source, a power switch coupled to the light source and the power supply and operable to control delivery of power from the power supply to the light source, and a light integrator adapted and configured to selectively transmit light from the light source to a target surface.

In still another aspect of the invention, a therapeutic treatment apparatus adapted and configured to conform to a surface of a patient is provided. The apparatus, or device, comprises a plurality of light sources flexibly interconnected to at least one other light source, a power supply coupled to the light sources and operable to provide power to the light sources, a controller coupled to the light sources and the power supply and operable to control the operation of the light sources, wherein each light source further comprises an optical waveguide adapted and configured to selectively distribute light onto the target surface. The waveguide can, in turn, be comprised completely or partially of silicone rubber. Additionally, the waveguide can further comprise one or more optical fibers.

In yet another aspect of the invention, a therapeutic treatment apparatus is provided that is adapted and configured to conform to a patient. The apparatus comprises a plurality of light sources adapted and configured to deliver light wherein the light sources are coupled to an elastomeric substrate and further wherein the substrate is comprised of a material having a durometer of less than or equal to shore 70 A and is at least partially transmissive to the light, a power supply coupled to the light sources and operable to provide power to the light sources, and a controller coupled to the light sources and the power supply wherein the controller is operable to control the operation of the light sources.

Another aspect of the invention is directed to a therapeutic treatment apparatus adapted and configured to conform to a target surface of a patient comprising: a plurality of light sources, a power supply coupled to the light sources and operable to provide power to the light sources, a controller coupled to the light sources and the power supply and operable to control the operation of the light sources, wherein the light sources are flexibly connected and further wherein the distance between at least two of the light sources is less than or equal to the distance between light sources and the target surface.

Yet another aspect of the invention includes a therapeutic treatment apparatus system comprising: a light source, a controller coupled to the light source, a power supply coupled to the light source and the controller and operable to provide power to the system, a fiber optic fiber adapted and configured to deliver light from the light source to a flexible substrate adapted and configured to conform to a patient's body surface, wherein the fiber optic fibers terminate into a light integrator which substantially uniformly distributes light onto target surface.

Still another aspect of the invention includes a therapeutic treatment apparatus adapted and configured to conform to a target region of a patient comprising: a plurality of light sources coupled to a flexible substrate, a power supply coupled to the light sources and operable to provide power to the light sources, a controller coupled to the light sources and the power supply and operable to control the operation of the light sources, and a light integrator adapted and configured to be positioned in at least a portion of an optical pathway between the light source and the target region of the patient, wherein the light sources are spaced such that D=2\sqrt{2dR−d²} where D is a width of light integrator, R is a radius of curvature of the target region, and d is a sum of tissue compression and an optically allowable gap between the light integrator and a target region.

Yet another aspect is directed to a therapeutic treatment apparatus adapted and configured to conform to a patient's body comprising: a plurality of light sources, a power supply coupled to the light sources and operable to provide power to the light sources, and a controller coupled to the light sources and the power supply and operable to control the operation of the light sources, wherein the light sources are adapted and configured to illuminate such that the light exiting the light source is substantially parallel with the body.

The invention also contemplates a method of treating a prescribed area of a target body surface. The method generally comprises the steps of applying a light therapy device adapted to conform to the target body surface; and selectively delivering a therapeutic dose of light to at least a portion of the target body surface. The method is suitable for treatment of clinical indications identified by a healthcare practitioner, such as psoriasis, vitiligo, atopic dermatitis, infection, sun tanning, acne, skin cancer, actinic keratosis, hair removal, dermal vascular lesions or pigmentation, skin rejuvenation, and bilirubin. As will be appreciated by those skilled in the art, these devices can be chilled prior to applying light therapy to a body, or during the delivery of light therapy.

Still another method contemplated is a method of treating a prescribed area of a target body surface comprising the steps of: administering a photosensitizer to a patient; applying a light therapy device adapted and configured to conform to the target body surface; and delivering a therapeutic dose of light to at least a portion of the target body surface.

Yet another method is directed to a method of treating a prescribed area of a target body surface comprising the steps of: applying a light therapy device adapted to conform to the target body surface and comprising a plurality of light sources; using a detector to determine at least one property of target tissue; and selectively activating one or more of the light sources in response to the detector to deliver a therapeutic dose of light to the target tissue. Additionally, the step of detecting can include detecting, for example, temperature, electrical impedance, photoreflectance, thickness, hardness, moisture, acoustic reflections. Additionally, measuring photo reflectance can include measuring one or more of: roughness, color, or fluorescence.

Another method of treating a prescribed area of a target body surface is provided that comprises the steps of: applying a targeting mask to the target body surface; applying a light therapy device adapted and configured to conform to the target body surface and at least partially coupled to the targeting mask; and delivering a therapeutic dose of light to at least a portion of the target body surface through the targeting mask.

Still another method of treating a prescribed area of a target body surface is provided that comprises the steps of: applying a substance to a non-prescribed region of a body surface which at least partially blocks therapeutic light; applying a light therapy device adapted and configured to conform to the target body surface to a prescribed region of the body surface and at least partially to the non-prescribed region; delivering a therapeutic dose of light to at least a portion of the prescribed region. As will be appreciated by those skilled in the art, light blocking substance can be, for example, cream, lotion, gel, ointment, paste, or fluid.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an example of a radiation applicator for applying radiation to a target surface;

FIG. 2A illustrates an example of target surface of a body being treated using the radiation applicator of FIG. 1;

FIG. 2B illustrates a cross-sectional view of a target surface of a body being treated using the radiation applicator of FIG. 1;

FIG. 3 illustrates a block diagram of an example of the radiation applicator of FIG. 1;

FIG. 4 illustrates a block diagram of a controller;

FIG. 5A shows a block diagram of an example of a radiation source used in FIGS. 1-3; FIG. 5B illustrates a cross section of a radiation applicator of FIG. 3; FIG. 5C illustrates another example of a radiation source of FIG. 1; FIG. 5D is a close-up of a molded covering with optical components built in; and FIG. 5E is a close-up of a mount with three-dimensional geometries optimized for radiation extraction from the source.

FIG. 6A illustrates yet another example of a radiation applicator; FIG. 6B illustrates an example of a cross-section of the radiation applicator of FIG. 6A; FIG. 6C illustrates a radiation applicator delivering radiation therapy to a prescribed surface area within a target body surface;

FIG. 7A illustrates a wearable optical therapy device in the form of a wrist bracelet; FIG. 7B illustrates an optical therapy device in the form of an adhesive bandage;

FIGS. 8A-B illustrates a planar light source that provides a uniform intensity of light;

FIG. 9 illustrates any of the embodiments of the invention adapted and configured to be placed on a target portion of a human body;

FIGS. 10A-C illustrates a flexible conformable light delivery device;

FIG. 11 illustrates depicts a cross-section of an embodiment of a flexible, conformable light delivery device according to the invention;

FIGS. 12A-B illustrate cross-sectional view of an interfacial feature between a plane of contact and a conformable, flexible light delivery device;

FIGS. 13A-B illustrate an embodiment of the invention where each light source is associated with an individual geometric region;

FIGS. 14A-C illustrate the building blocks of a geometric region;

FIGS. 15A-C illustrate schematic plan view of embodiments of single cell geometry;

FIG. 16 is a schematic representation of a design rule that illustrates a relationship of various design components for embodiments of the invention;

FIGS. 17A-E illustrate various views of an embodiment of the invention;

FIG. 18A-B illustrate a light source associated with a housing;

FIGS. 19A-B illustrate variations of a light housing, light source element;

FIGS. 20A-B illustrate an exploded view of a view according to the invention, and the configured device;

FIGS. 21A-C illustrate a masking element;

FIG. 22 illustrates an alternative embodiment of a device for delivering targeted phototherapy;

FIG. 23; depicts a detailed view of a module which is insertable into a radiation applicator;

FIG. 24 depicts a schematic view of several component elements in an example computer simulation of an optical intensity distribution.

FIG. 25A-B depicts the resultant intensity profiles calculated in the example simulation.

### DETAILED DESCRIPTION OF THE INVENTION

A radiation applicator used for irradiating a target portion of a body for medical treatment is disclosed. In an embodiment, radiation delivered by a radiation applicator is ultraviolet light. In other embodiments, other forms of radiation may be delivered by the radiation applicator.

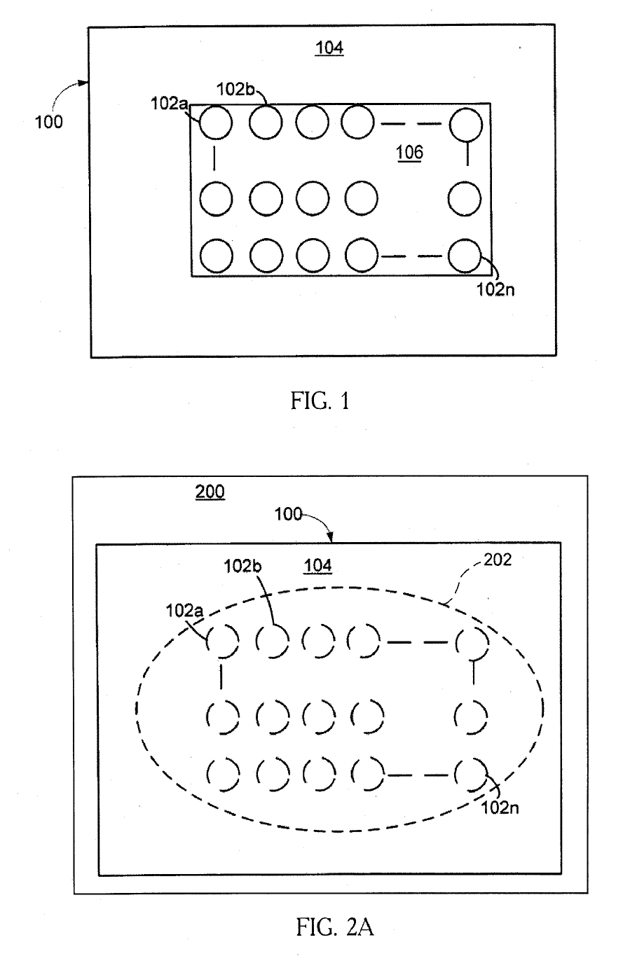

FIG. 1 shows a radiation applicator 100 for treating a target surface of a body with radiation. As will be appreciated by those skilled in the art, the target surface of a body includes the portion of a body surface onto which a radiation applicator is applied when the device is deployed for use on the target surface of the patient's body. At least a portion of the target body surface will include an area to which radiation therapy will be applied, such as a lesion. The portion of the target body surface to which radiation therapy is applied can, for example, be referred to as the therapeutic surface area or the prescribed surface area. As will further be appreciated by those of skill in the art, the therapeutic surface area can be of a size and shape that may or may not be conformable with the size and shape of the area comprising the target body surface. Thus, the size and shape of both the therapeutic or prescribed surface area can be the same, or substantially the same, as the size and shape of the target body surface. Alternatively, the size and shape of the therapeutic or prescribed surface area can be smaller or larger than the target body surface, without departing from the scope of the invention.

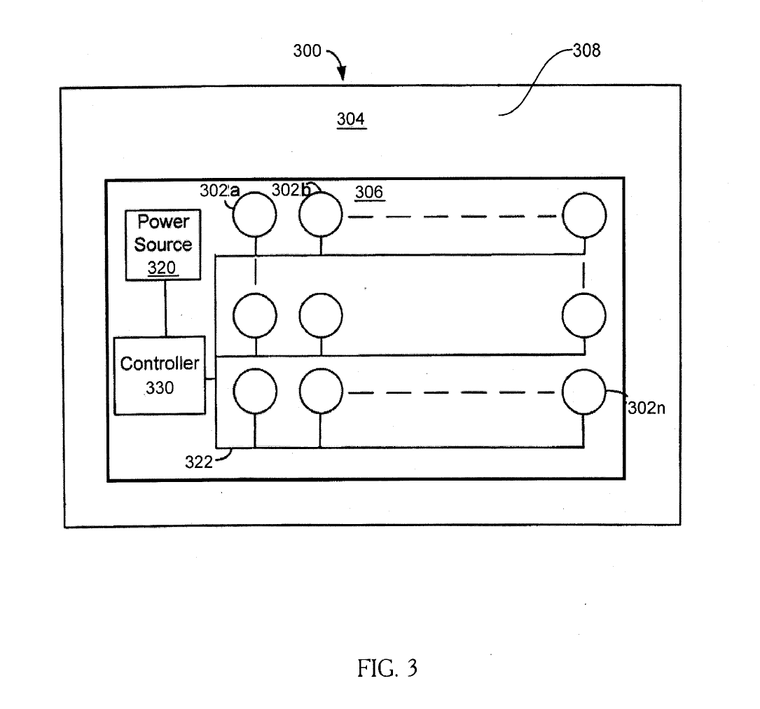

The radiation applicator 100 has at least a first side and a second side, or a top side and a bottom side with one side applied to the target body surface while the other side, typically, is not. The target surface is typically an exposed portion or surface, e.g. of skin, where it is desirable to apply radiation. Radiation applicator 100 may include one or more radiation source(s) 102 (e.g. 102a-102n) each of which has at least a first side and a second side, and substrate 104, also having a first side and a second side, which can be in the form of a layer or material on which the electrodes are formed or fabricated. In a preferred embodiment a plurality of radiation sources 102 are provided. Radiation sources refers to the actual source of the radiation and can also include structural elements associated with the source of energy which allow the radiation source to be manipulated independently of the substrates and other radiation sources. For example, (as discussed below) in the case where the radiation source is a light source, radiation source 102 can include a header, electrodes, reflecting features, focusing features, mounts with circuits and/or heat transferring features included thereon, and submounts. In further embodiments, the radiation applicator 100 has a delivery region 106 that has a surface area smaller than the surface area of the substrate 104 (as illustrated in FIG. 3). As will be appreciated by those skilled in the art, radiation applicator 100 need not have all of the components depicted in FIG. 1 and/or may include other components in addition to or instead of those depicted with FIG. 1. For purposes of illustration, the geometric profile of the radiation applicator 100 has been shown as having a rectangular profile (e.g. a length greater than a width). As will be appreciated by those skilled in the art, other profiles can be employed, either geometric or non-geometric (e.g., random) without departing from the scope of the invention. The various layers and elements of the applicator 100 can be configured such that each provides a surface-to-surface contact with an adjacent layer and/or element.

Radiation source(s) 102 may produce any of a variety of types of radiation, such as UV light, white light, and/or infrared light that are used for treating disorders, ailments or diseases by irradiating a target portion of the body, such as an exposed surface of skin. A variety of dermatologic conditions, such as psoriasis, contact dermatitis, atopic dermatitis, vitiligo, seborrheic dermatosis, acne, cellulite, unwanted hair, unwanted blood vessels, and skin cancer, may be treated with various wavelengths of light, as discussed above. For example, when treating psoriasis, radiation source(s) 102 may emit light having a wavelength in the UVB range, including 295-320 nm, 300-305 nm, 308-315 nm, or a combination of these wavelengths in one or more peaks. When treating psoriasis with psoralen (PUVA), it is desirable to use radiation sources which emit light in the UVA range, for example, between 320 nm and 340 nm, between 341 nm and 360 nm, and/or between 361 nm and 390 nm. Additionally, there may be any number of radiation source(s) 102 with any combination of wavelengths.

It may be desirable to provide radiation source(s) that are capable of delivering more than one type of radiation. For example, atopic dermatitis can be treated with a device using, for example, a combination of UVB and UVA wavelengths. Thus, alternatively, it may be desirable to provide radiation source(s) 102 within the substrate 104 that can deliver a first radiation type or wavelength in combination with radiation source(s) 102 that can deliver a second, or subsequent, radiation type or value that is different from the first radiation type or wavelength. As will be appreciated by those of skill in the art, additional wavelengths or sources of radiation can be included without departing from the scope of the invention, and thus the invention is not limited to the delivery of two radiation types.

Infectious disorders can also be treated with the radiation source(s). For example, where infectious disorders are treated, shorter wavelengths, including those having a wavelengths in the range 254-270 nm or 270-295 nm, have been shown to be beneficial. As will be appreciated, the various dashed lines between various ones of radiation source(s) 102 (e.g. 102a-102n) indicate that there may be any number of radiation source(s) in that location spanning the region of the dashed lines and the region between the dashed lines, as necessary or desirable.

In another embodiment, radiation source(s) 102 (e.g. 102a-102n) produce white light (500-750 nm), infrared light, microwaves, radiofrequency radiation, and/or other electromagnetic wavelengths, for example, or combinations thereof. Heat (via infrared light) sometimes promotes healing of sprains and muscle injuries, and additionally may produce a feeling of well-being, even if no actual healing occurs. Infrared wavelengths include wavelengths from 780 run to 10 microns. Infrared light can also be used to aid in healing of open surface wounds on a body or to increase the blood flow to a body surface. In some embodiments, the infrared light can be used to increase local blood flow to a body surface in order to improve the efficacy of phototherapy or photodynamic therapy. In some embodiments, infrared light can be used to destroy hair follicles which results in permanent or semi-permanent hair removal; cellulite can also be treated with infrared wavelengths. Other wavelengths of light in the mid-visible range (e.g. about 500-650 nm) can be used to treat acne, wrinkles, or other undesirable spots; white light wavelengths can also be used for photorejuvenation and/or cellulite removal. Some wavelengths of light (e.g. those having a wavelength of 450-460 nm) may be effective in treating different disorders, such as for lowering the bilirubin count in babies. In one embodiment, radiation source(s) 102 are used for treating disorders on a surface of a body. In another embodiment, radiation source(s) 102 emit forms of radiation (e.g., wavelengths of light) that penetrate below the surface of the body, and radiation source(s) 102 are used for treating disorders below the surface of the body. In some embodiments, some of radiation source(s) emit forms of radiation that penetrate to difference levels than other of the radiation source(s) 102. In some embodiments, photodynamic therapy is initiated with radiation source(s) 102. Photosensitizers allow for the application of almost any wavelength. For example, a photosensitizer can be applied to a skin lesion, and then the radiation device can then be applied over the lesion for a long period of time, for example by bringing the device into nearness or contact with the skin, or by putting the device on the skin, where the time is sufficient for a requisite dose of radiation to treat the lesion. In the case where the device is portable, a patient does not have to wait in a physician's office and a physician does not have to spend valuable time manually applying a tedious treatment. Photodynamic therapy can include a portable light source (e.g. device 100) and a photosensitizer which can be administered systemically or injected into a lesion or placed in close proximity to the lesion (e.g. a cream). For example, the photosensitizer can be applied and then the radiation applicator applied to the area over time to activate the photosensitizer. Alternatively, the radiation device releases photosensitizer from a reservoir or from the substance of the device itself. For example, levulin is a photosensitizer used in combination with yellow light for photorejuvenation therapy.

In one embodiment, all radiation source(s) 102 produce the same peak wavelength and/or spectrum of radiation when activated. In another embodiment, different ones of radiation source(s) 102 produce different spectrums of radiation and/or have different peak wavelengths. In an embodiment, whether or not all radiation source(s) 102 are the same or some are different from others, the spectrum of radiation produced may be controllable (e.g., by adjusting the current) so that the wavelength or combination of wavelengths of light may be adjusted according to the type of disorder being treated. In some embodiments where an optical disperser is used, a multiplicity of radiation source(s) can be combined into a predetermined spectral output. In these embodiments, the spectrum can be tailored by turning one or more of the radiation sources on or off at different times.

Radiation source(s) 102 may require a power source. Embodiments including a power source are discussed, for example, in conjunction with FIGS. 3, 5C, and 6A, for example. Power sources may be portable (e.g. wearable or incorporated into the device, etc.) or non-portable (e.g. table top, wall-plug, or other wise connected to the device via cord, etc.) Alternatively, some radiation source(s) 102 may not require a power source. For example, radiation source(s) 102 may produce light via fluorescence or chemical luminescence. In another embodiment, radiation source(s) 102 can be powered by photovoltaic cells. Alternatively, radiation source(s) 102 may include a radioactive material that emits alpha, beta, and/or gamma particles. For example, radiation source(s) 102 may be discs of P-32, In-111, radioactive isotopes, Cesium 137 and/or another radioactive material, which may be useful for treating certain types of cancer. Additional radiation sources can include microwave emitters, electromagnetic emitters, and radiofrequency emitters.

Substrate 104 may take many forms. Substrate 104 may be any suitable material such as a piece of material, which in turn may be a strip of fabric. Substrate 104 may be solid, a mesh, or netting, for example. Substrate 104 may be a flexible material that can be wrapped around a limb or placed on another body part. In one embodiment, substrate 104 is a bandage. For example, substrate 104 may have an adhesive layer on at least a portion of one surface of the substrate such as the surface that contacts the target body surface. Alternatively, substrate 104 does not have an adhesive layer. In another embodiment, substrate 104 may be an article of clothing, such as a sock, a glove, a sweater, a ski mask, a headband, an arm band, a leg band, etc. In some embodiments, the substrate 104 is patient compatible. If substrate 104 is not patient compatible, then the substrate can be furthered covered with a patient compatible material. As will be appreciated by those skilled in the art, substrate 104 can be any material, surface or device adapted and configured to deliver radiation therapy to a body surface. Thus, the radiation therapy device can be configured to delivery therapy such that the device is a therapeutic treatment apparatus.

In another embodiment, instead of being flexible, substrate 104 is rigid and is held onto the portion of the body being treated by being attached to a bandage or by being wrapped within a bandage. Whether substrate 104 is rigid or flexible, a separate substrate, such as a stocking, a glove, or a circumferential cloth, may be utilized to hold the substrate 104 onto a target portion of a body.

Substrate 104 may be opaque, transparent, translucent, reflective, or made from a light scattering material. Radiation source(s) 102 (e.g. 102a-102n) may be located on substrate 104. For example, radiation source(s) 102 may be attached to a surface of substrate 104 and/or formed integrally within substrate 104 (e.g., embedded or formed within the substrate to provide a complete, unified radiation applicator 100). Alternatively, one portion of the radiation source can be attached on the outside of the material (e.g. the side of the material not facing the lesion or target body surface) and the other side of the radiation source (e.g. the light emitting side) is attached on the inside of the substrate (e.g. the side of the material facing the lesion). In this embodiment, the housing of the radiation source traverses the substrate 104 and the power is supplied along the surface of the substrate 104 facing away from the region of the body with the lesion. Substrate 104 may be of a size and/or shape that facilitates securely attaching radiation applicator 100 to a body. In an embodiment, radiation applicator 100 can be worn by a patient without any external attachments. In an embodiment, radiation applicator 100 may be self-contained. Making radiation applicator 100 self-contained and/or wearable without any external attachments (e.g., in the form of an adhesive bandage) facilitates making radiation applicator 100 portable. A portable applicator which can be worn by a patient under other clothes or while he or she is performing other tasks or while sleeping may have many advantages in terms of, for example, the quality of life of the patient and in terms of compliance.

Region 106 is a region of substrate 104 within which radiation source(s) 102 (e.g. 102a-102n) are located. Region 106 can have a surface area that is less than the surface area of substrate 104. Substrate region 106 may be of a size and/or shape that is expected to cover all of, or a substantial part of, a portion (of a body) affected by a typical occurrence of a particular type of disorder (such as a lesion). Alternatively, region 106 may be of a size and/or shape that is expected to be smaller than the portion of the body affected by a typical occurrence of a particular type of disorder. In one embodiment, substrate region 106 is defined only by the location of radiation source(s) 102, but is otherwise structurally identical to the rest of substrate 104. In another embodiment, region 106 may have one or more structural features that distinguish region 106 from the rest of substrate 104. In one example, substrate 104 is rectangular in shape, optionally having rounded corners, and region 106 is located in a central portion of substrate 104 that extends nearly the entire width of substrate 104, but only extends less than one third or less than one quarter of the length of the substrate 104. In a further embodiment of this example, substrate 104 is flexible and has an adhesive in the portions 108 outside of the region 106 for adhering to a body being treated, but no adhesive is inside of region 106. Region 106 may be analogous in structure to the gauze pad of a Bandaid® type bandage. In this example, region 106 and substrate 104 are of a similar size as the gauze pad region of a bandage for covering a cut or scrape. For example, region 106 may include a gauze pad, and any one of, any combination of, or all of radiation source(s) 102, controller 320 (discussed below), and/or power source 330 (discussed below) may be located on, behind, and/or embedded within the gauze pad.

As will be appreciated by those skilled in the art, the controller can be adapted and configured to control the delivery of radiation either automatically (i.e., without user intervention) or semi-automatically (with minimal or limited user intervention). The controller can be adapted and configured to control the amount of radiation delivered, the time for which radiation is delivered and the type of radiation delivered. Further, the controller can be adapted and configured to provide a therapeutic regimen, e.g. by altering or changing the type and/or amount of radiation delivered. The controller, or suitable electronic circuitry, can also be adapted to dynamically control the operation of the light sources and to further control the therapeutic regimen delivered in response to feedback, as will be appreciated based on the teachings herein.

Substrate region 106 may include a protective layer for radiation source(s) 102 that is not present in the remainder of substrate 104. Within region 106, substrate 104 may have additional elements or features, such as structural features, that promote cooling, or condition the spectral output of radiation source(s) 102; for examples substrate 104 can contain a deposited reflective layer such as aluminum in the case of UV light. Alternatively, substrate 104 contains surface features which increase the surface area to promote heat transfer. Other elements and features include, but are not limited to, selectively providing perforations (not shown) that penetrate all or a portion of the radiation applicator 100 on at least a portion of the applicator. In yet another embodiment, region 106 may be a piece of removable material that supports radiation source(s) 102. Having a removable substrate region 106 allows the same substrate 104 to be used with a multiplicity of different sets of radiation source(s) 102 in which each set is designed for treating a different disorder or set of disorders. In another embodiment, a material covers region 106. This material is a disposable material which is transparent to the radiation from radiation source(s) 102 and is discarded after the therapy, allowing the devices in region 106 to be reusable without concern for the devices being soiled. In another embodiment, substrate region 106 may be absent, and radiation source(s) 102 may be uniformly distributed throughout substrate 104.

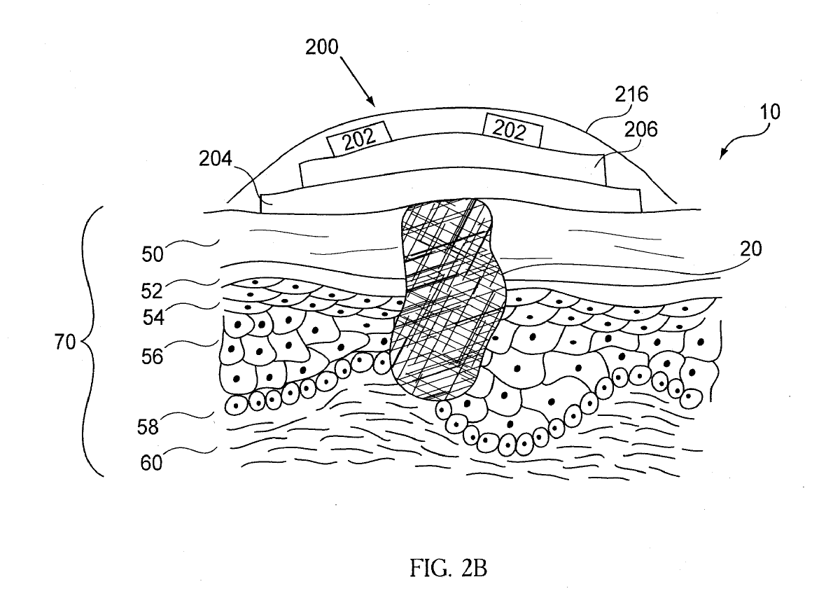

FIG. 2A shows an example of a portion of a body 10, e.g. a target portion of a human body, such as a skin layer, while being treated. During treatment of body portion 10, radiation applicator 200 is placed on a lesion 20 on body portion 10. Lesion 20 can be any patch of unhealthy or unwanted tissue surface that is expected to be at least partially treatable by irradiating with radiation, such as light. (Lesion 20 is illustrated with a dashed line in FIG. 2A because lesion 20 is under radiation applicator 200 and specifically under region 206.) Body portion 20 is any target external surface of a body, e.g., skin. For example, portion 20 may be a portion of skin on a limb (e.g., the arm), or the hand of a patient. In the embodiment of FIG. 2A, substrate 204 is a single opaque layer and radiation source(s) 202 (e.g. 202a-202n) are placed on one side of substrate 204. Consequently, radiation source(s) 202 (e.g. 202a-202n) are drawn with dashed lines to indicate that radiation source(s) 202 are between substrate 204 and lesion 20, so as to irradiate lesion 20 without being impeded by substrate 204. Similar to FIG. 1, the various dashed lines between radiation source(s) 202 indicate that there may be any number of radiation source(s) in that location spanning the region of the dashed lines and between the dashed lines. Although FIG. 2A illustrates an embodiment in which substrate 204 is a single opaque strip, any of the other embodiments of radiation applicator 200 may be used instead.

If substrate 204 is transparent or translucent to the radiation source(s) 202, then substrate 204 could be placed between radiation source(s) 202 and lesion 20. An advantage to placing substrate 204 between radiation source(s) 202 and lesion 20 is that radiation source(s) 202 may be left exposed to air, which may facilitate passive and/or active (e.g. a thermoelectric cooling device) cooling of radiation source(s) 202. Additional structural elements such as fins or other heat diffusing, heat dispersing, and/or heat sinking elements can be attached or manufactured on substrate 204; additionally, electrodes or other conductive paths can be applied to or manufactured on substrate 204. Processes such as chemical or vapor deposition processes can be used to deposit heat conducting or electrically conducting materials on substrate 204. Alternatively, the radiation source(s) 202 may be adapted to traverse the material so that the light emitting face is placed between the substrate 204 and lesion 20 and the electrical connections and heat generating components are such that they direct heat away from the lesion 20 (and/or electricity toward the radiation source(s) 202) through the substrate 204, and then to the ambient atmosphere. Also, substrate 204 may include elements and/or structural features that facilitate uniform irradiation of lesion 20, such as by scattering or focusing the radiation emitted from radiation source(s) 202. One example of a scattering structure is a substrate having one or both of its outer surface and its surface facing radiation source(s) 202 roughened or textured. Another example of a scattering structure is a substrate having particles (e.g. titanium oxide and/or aluminum oxide) embedded within it that have a different index of refraction than the substrate. Any one of, any combination of, or all of these scattering structures may be included in substrate 204 (and/or within other layers) for uniformly irradiating lesion 20.

An advantage in placing radiation source(s) 202 between substrate 204 and lesion 20 is that a greater percentage of the radiation generated is incident upon lesion 20. Consequently, the power efficiency may be greater without substrate 204 intervening between radiation source(s) 202 and lesion 20 than with substrate 204 in an intervening position.

FIG. 2B illustrates a target body surface, such as a layer of skin 70. The layer of skin is comprised of the stratum corneum 50, the stratum lucidum 52, the stratum granulosum 54, the germitive layer 56, 58 and the dermis 60. Lesion 20 is depicted crossing all of the layers for purposes of illustration. However, as will be appreciated by those skilled in the art, the layers of the skin affected by the lesion will be determined by the type and extent of medical condition associated with the skin, e.g. psoriasis, contact dermatitis, vitiligo, acne, atopic dermatitis, cellulite, collagen laxity associated with aging, and skin cancer. In this illustration, the radiation applicator 200 is positioned on the target body surface to be treated such that the radiation source(s) 202 will be in proximity to the lesion 20. As described above and below, the radiation applicator can contain a multitude of radiation generators which alone or in combination can apply radiation to difference depths within the lesion. For example, infrared wavelengths can be used to penetrate the deeper parts of the lesion whereas ultraviolet wavelengths can be used to penetrate the more superficial portions of the lesion. Photosensitizers can further be utilized to modulate the depth of penetration. For example, if a red light absorbing photosensitizer is applied superficially to the lesion, then the superficial portion of the lesion is treated with the red light. In this embodiment, the depth wherein light activates the photosensitizer is determined by the depth where the photosensitizer is placed or level it is absorbed to. If the photosensitizer is injected 2 mm underneath the skin, then the light will be absorbed in this layer assuming that light is not absorbed in the more superficial layers of the skin.

FIG. 3 shows a block diagram of an example of radiation applicator 300. Similar to FIG. 1, FIG. 3 shows radiation source(s) 302 (e.g. 302a-302n), substrate 304, and region 306. Additionally, FIG. 3 shows controller 320, power source 330, and electrical connectors 322. In other embodiments, radiation applicator 300 may not have all of the components associated with FIG. 3 and/or may have other components in addition to, or instead of, those depicted for purposes of illustration with FIG. 3. Radiation source(s) 302, substrate 304, and region 306 were described in conjunction with FIGS. 1 and 2A-B. Controller 320 may function as an on/off switch. Controller 320 may include a processor and/or a specialized circuit for controlling radiation source(s) 302. Controller 320 may be a microcontroller. For example, controller 320 may have a width and/or length that are less than 5 cm, less than 4 cm, less than 3 cm, less than 2 cm, or less than 1 cm. As discussed above, controller 320 can be adapted and configured to control radiation source(s) 302 and may control how long and/or which ones of radiation source(s) 302 is/are powered on. Additionally, or alternatively, controller 320 may control the wavelength, frequency, and/or the intensity of the radiation of radiation source(s) 302. In addition, controller 320 can integrate feedback from reflectance sensors (not shown) associated with the device 300 which relay real-time information about the state of the lesion or of the surrounding skin. Controller 320 further has the ability to be programmed from a device (e.g. a wireless or wired device such as a computer, personal digital assistant, etc.) outside the radiation applicator 300. A therapeutic treatment may be provided where the specific areas of a patient's body surface considered to be affected (for example, containing a plaque or lesion) substantially receive a majority of the dose provided by the delivery device. In an embodiment, there is at least one sensor located on the device that can provide feedback to an operational controller 302. This sensor (not shown) has the ability to detect the physical condition of a particular area of a patient's body surface. This physical condition can be evaluated by assessing one or more of the following characteristics: photoreflectance, temperature, electrical impedance, hardness, thickness, moisture, or acoustic reflections, among others. Photoreflectance may measure roughness, color, fluorescence, or other characteristics. The controller can process the information and determine whether or not the particular body surface may receive radiation. As an example, based on input from the previously described sensor, controller 302 can determine whether or not a particular segmented region of a body surface contains an affected area or not. In practice, the device described herein may be placed on a body surface containing areas that are in need of treatment along with areas that are considered healthy and otherwise not requiring therapy. Therefore it would be entirely beneficial to enable sensing of the specific location of, for example, a psoriasis plaque residing on a body surface within the periphery of device 100. Subsequent to this detection step, a therapy can selectively be applied only to the plaque region. This can be accomplished by selectively enabling the one or more radiation source(s) 102 with respect to a particular area on such a body surface.

In an embodiment, controller 320 may relieve the patient and/or doctor from the task of keeping track of the time that the therapy has been applied. For example, controller 320 may track the total amount of time that each individual one of radiation source(s) 302 and/or each of a plurality of groups of radiations source(s) 302 has been in use. In other words, each of radiation source(s) 302 may be turned on and off in cycles, and controller 320 or a timer (not shown) may keep track of the total amount of time and/or total energy that any given radiation source(s) has been kept on. The controller in some embodiments facilitates the portability of the device. If the dosage being applied to the patient is not being monitored by the physician or the patient it would therefore be possible that too high a dose is delivered to the treatment area. With a controller 320 various groups of radiation source(s) 302 may be turned on and off together, separately or not at all while keeping track of how long an individual radiation source has been on and/or how long a group of radiation source(s) associated with this individual radiation source has been on, (because the group of radiation source(s) and any individual radiation source within the group is expected to have been on for the same amount of time). In some embodiments, the device is provided with a computer interface so that the patient or doctor programs the computer interface and subsequently the device to achieve a specific dose on one or more target areas. For example, the user of the computer interface determines the region to be treated and the dosage to be applied. This methodology ensures that a specific dosage is applied to a specific (e.g. diseased) location on the body surface. In this way, the ideal toxicity: efficacy ratio can be obtained.

When a particular one of, or group of, radiation source(s) 302, has delivered a predetermined therapeutic dose of energy, radiation controller 320 turns off or otherwise decreases its applied dose 302. A therapeutic dose of radiation may be an amount of radiation that has been determined to be the maximum or slightly less than the maximum tolerable dose during a particular treatment session. Tolerable can mean a sunburn in the case of ultraviolet light applied to the skin. Alternatively, a therapeutic dose of radiation may be an amount of radiation that has been determined to be appropriate for a particular disorder or a particular treatment session. As will be appreciated by those skilled in the art, different disorders may have different therapeutic doses. For example, a therapeutic dose may be a sub-threshold Minimal Erythemal Dose (“MED”) in some skin disorders. As another example, a therapeutic dose may be reached when all the radiation source(s) 302 or when all of the groups of radiation source(s) 302 have delivered 100-600 mJ/cm² (of ultraviolet light in the 295-320 nm range for example) to body portion. Consequently, when all of the groups of radiation source(s) 302 have delivered 100-600 mJ/cm² to portion, the therapy for that region is finished.

Although FIG. 3 shows an example in which there is only one controller 320, there may be a plurality of controllers. Each one of radiation of sources 302 or each group of radiation source(s) 302 may have its own controller. There may be a system of controllers in which there is one master controller that controls other local controllers, and the local controllers may control individual ones of and/or groups of radiation source(s) 302. Optionally, controller 320 may have one or more input ports or input devices that may be used for programming, inputting parameters, and/or setting controller 320 according to a particular therapy, which may be based on a calibration that was performed. The programming, input parameters, and/or settings may be entered by a patient, entered by a doctor, and/or automatically entered as part of a calibration and/or setup procedure. Examples of inputs include, but are not limited to, Bluetooth®, USB, optical, or any other wired or wireless connections.

Power source 330 powers controller 320 and/or radiation source(s) 302 are provided for as shown in FIG. 3. In the example of FIG. 3, power source 330 supplies power to radiation source(s) 302 via controller 320. Power source 330 may be one or more batteries, a power supply that plugs into an outlet, and/or one or more photocells for recharging one or more batteries. Power source 330 may include one or more flat, disc-shaped batteries, which may be less than 2 or 3 millimeters thick, and less than 1 or 2 centimeters in diameter. For example, power source 330 may be one or more lithium ion batteries. Alternatively, power source 330 may be one or more nickel cadmium, AA, and/or AAA batteries, for example. Although in the example of FIG. 3 there is only one power source shown, there may be a plurality of power sources located in a plurality of locations within radiation applicator 100. Each one of, or each group of, radiation source(s) 302 (e.g. 302a-302n) may have their own power source. Power source 330 may be located on substrate 304. In an embodiment, power source 330 is an integral part of substrate 304 (e.g., power source 330 may be embedded within substrate 304). In another embodiment, power source 330 is one or more photovoltaic cells.

Depending upon the configuration of the radiation applicator 300, the weight of the device can range from, for example, 0.5 g to 200 g, more preferably from 0.5 g to 100 g, and even more preferably from 0.5 g to 10 g. As will be appreciated by those skilled in the art, these weight ranges are meant to be illustrative of a reasonable weight which an individual can tolerate. Other weight ranges could be used without departing from the scope of the invention.

Electrical connections 322 communicatively connect radiation source(s) 302 (e.g. 302a-302n) to controller 320 so that controller 320 is capable of controlling radiation source(s) 302. Electrical connections 322 also electrically connect power source 330 to radiation source(s) 302, via controller 320, such that power source 330 supplies power to radiation source(s) 302. Electrical connections 322 may include a bus that sends signals to individual radiation source(s) 302. Alternatively, electrical connections 322 may include individual pairs of electrical connections, where each pair links one of, or one group of, radiation source(s) 302 directly to controller 320.

Electrical connections 322 may be attached to substrate 304 individually or they may be created directly on the material by a process of photolithography, electrodeposition, chemical vapor deposition, and/or physical vapor deposition. Alternatively, electrical connections 322 are embedded in a flexible insulating film, the entire film then being attached to substrate 304. Electrical connections 322 can be wire-bonded connections produced using a wire bonding process well-known in the LED arts. These connections are three dimensional and can be protected via material film around the connections. One representative example of a flexible film is a silicone film. A silicone film can be used to embed wires which lead to a connector such as a computer pin connector. After the bus and the wires are embedded, the film can be mated with another film which is a radiative device or a heat conducting film. When the two sides (film with the wires and film with the LEDs) are mated to one another, the device is electrically connected.

A method of applying radiation therapy in the context of this invention includes the steps of: visualizing a body surface to be treated; mapping the body surface to be treated in a device interface; delineating an area of the body surface to apply radiation therapy to; programming a topologic dosage map to the radiation therapy device via the computer interface; applying the radiation therapy device to the body surface in an orientation where the topologic dosage map align with the underlying disease being treated; and allowing the radiation therapy device to function autonomously after the device applied to the body surface.

In some embodiments, doses are applied to the treatment region on a continuous basis and the maximum therapeutic dose guides the therapy. For example, a time can be defined, over which a maximal dose cannot be exceeded. Using the skin as an example, an MED, a fraction of an MED, or a multiple of an MED can be given to a body region over a 30 second period, a 12 hour period, a 24 hour period, a 48 hour period, or over any period of time in between or other time chosen by the patient or the physician; it is also conceivable that erythema (in the skin for example) can be avoided altogether when the dose is given over a long period of time. After this period of time, another dose is give to the same region or another region. In other embodiments, the dose delivered to the region with the lesion can exceed the toxicity dose of the non-lesional region because the radiation device can selectively apply radiation to one region versus another region and the application region can be programmed into the device by the physician or the patient. For example, in the case of psoriasis, the dose that can be delivered to the region with a psoriatic plaque can exceed the minimal erythemal dose by a factor of, for example, 2,3,4,5,6,7,8,9, or 10 because the psoriatic region is more resistant to radiation than normal skin. With most existing devices, it is not possible to define a treatment region while avoiding non-treatment regions. It is typically the responsibility of the operator of the device to apply radiation to unhealthy regions and not healthy regions.

In an embodiment of the method, a radiation applicator, such as applicator 100, may be programmed by the patient or by the physician to deliver a particular therapy over a period of time. In an embodiment, controller, such as controller 320, may be programmed to calibrate radiation applicator or have a calibration mode during which radiation applicator is calibrated. For example, radiation applicator may be calibrated for the patient prior to applying a therapy (e.g. due to the fact that different patients have different sensitivities to light due to differing amounts of melanin contained in a patient's skin).

During calibration, radiation applicator is placed on a portion of the body that is unaffected by the disorder that portion is affected by. For example, radiation applicator is placed on a portion of healthy skin typically unexposed to sunlight (e.g., the gluteal region). Next, escalating doses of radiation are applied to the skin. The dose, which after 24 hours produces a superficial redness of the skin from dilation of the capillaries, or erythema, is called the Minimal Erythemal Dose (MED). Controller may be programmed to automatically apply the escalating doses to different regions under radiation applicator. After 24 hours, the MED is determined by the region which has a perceptible erythema, or redness. The patient's MED is then programmed into controller and the MED, or an amount of radiation slightly less than the MED, becomes the calibrating dose for the particular patient. This device configuration can also be utilized to diagnose disease. For example, the disease state, polymorphic light eruption, is a disease in which an allergic response occurs with light exposure. It is typically a tedious process to diagnose the specific wavelengths and/or power required for the allergic response to light, requiring a large amount of technician time and equipment. A radiation device 100 can be used for diagnosis in some embodiments. For example, radiation device can have a multitude of radiation sources with different wavelengths, each of which deliver specific energies in different wavelength bands. The radiation device can then be applied to a body surface (e.g. skin) with a program to deliver a specific wavelength and/or dose to different body surface areas under the device over specific times. After the doses are delivered, the region which develops the skin reaction can be determined by observing the region which has the reaction. Similarly, a radiation device can be used to determine body reactions to photosensitizing pharmaceuticals, cosmetics, natriceuticals, and sunblocks. In the case of sunblocking compounds, various compounds can be placed underneath the radiation device and prescribed doses of radiation programmed into the device. The radiation applicator in these diagnostic embodiments can further be adapted to fit animals, such as pigs, rats or mice which are often used to test the potential photosensitizing compounds.

To treat a disease such as psoriasis, doses are typically related to the MED. For example, a standard course of therapy consists of 3 weeks of treatments, 3 times per week, with each treatment consisting of 1-3 MED depending on what the patient can tolerate. It is difficult, if not impossible, for the treatment area to be well-controlled; some areas of non-diseased skin will receive treatment. It is these areas which limit the amount of radiation which the affected areas can receive. Further, the risk of skin cancer is increased in the areas unaffected by disease but which are nonetheless exposed to radiation because of the non-specificity of the radiation applicator. Furthermore, the treatments are given three times per week solely because the unaffected skin must heal before the next treatment. A device which could limit treatment area to the lesional area could be beneficial in that the treatment dose and/or frequency could be increased and the total treatment time decreased. Furthermore, a device which does not require the patient to be at the physician's office or otherwise schedule time for a treatment could be highly beneficial in many patients and result in greater treatment protocol compliance by the patient which in turn would lead to greater efficacy of patient treatment. With radiation applicator, the treatment region can be finely tuned by the patient and/or physician. In embodiments where the device is worn by the patient, the patients do not have to stop what they are doing (e.g. work, sleep, exercise, etc.) to receive treatments.

In embodiments in which controller is kept small (e.g., in embodiments in which controller is a microcontroller), the small size facilitates making radiation applicator portable. Controller may be located on substrate. In an embodiment, controller is an integral part of substrate (e.g., controller may be embedded within substrate). Controller switches power between different radiation source(s), so that some of radiation source(s) are powered on while others are powered off. In an embodiment, controller may never, or only infrequently, power on all of radiations sources simultaneously. Alternatively, controller will have at least some period of time when not all of radiation source(s) are powered on simultaneously. If controller does not keep at least some of radiation source(s) (although not necessarily the same radiation source(s)) off all of the time, nearly all of the time, most of the time, or at least some of the time, the current required for operation may be very high and may generate excess heat in addition to requiring a very large power source as compared to the operating current required, the heat generated, and the size of the power source when some or all radiation source(s) are turned on and off to conserve power. A large power source and excessive heat dissipation requirements may require component sizes that limit the portability of a radiation applicator and the ease and/or comfort with which radiation applicator can be worn. The selective activation of radiation source(s) and the duration of radiation source activation time (e.g., the duty cycle) may be based upon the power capacity of a power source, which is kept small enough to keep radiation applicator portable and self-contained. Alternatively, or in addition to, the amount of time that a given one of radiation source(s) is kept on may be based upon cooling considerations and/or a desired intensity of radiation that is expected to be therapeutic. In an alternative embodiment, radiation applicator is connected to an external computer or an external controller during, before, or after operation or is at least in part controlled wirelessly by a remote unit during, before, or after treatment. Additionally, as will be appreciated, the power source may be contained in a water-resistant or water-proof housing (not shown). The housing may be configured to be connectable to the radiation applicator in such a manner that the connectors between the radiation applicator and the housing can be connected in a manner that provides a secure moisture resistant connection.

Using a microcontroller for controller may simplify the structure of the radiation applicator as well. For example, in an embodiment in which each of radiation source(s) (e.g. 102a) is on for only a short period of time before being turned off and another one of radiations sources being turned on, heat transfer through substrate is not as large an issue as it would be if all of radiation source(s) were run continuously. Consequently, there may not be any need to pump a fluid through radiation applicator for cooling. Similarly, there may not be any need for perforating substrate for cooling.

Optionally, radiation applicator may include one or more detectors to detect whether the body surface of the patient has been harmed and/or may be harmed soon. For example, radiation applicator may include one or more detectors to detect erythema. The detectors may detect erythema by detecting the color of a target portion of the body or a change in the color of a target portion of the body (e.g., skin color). In another embodiment, there may be detectors for detecting the color, moisture, and/or temperature of the target portion being irradiated to ensure that the portion irradiated is not being damaged by the radiation. Optionally, after detecting erythema and/or any other condition indicative that radiation applicator may have harmed, or may harm, the target portion being irradiated, controller may automatically turn off radiation source(s). Controller may turn off the radiation source(s) associated with the erythemal region as part of the calibration routine and/or as a safety feature during a treatment in response to input from one or more detectors concerning the condition of the region being irradiated (e.g., after an erythemal condition is detected).

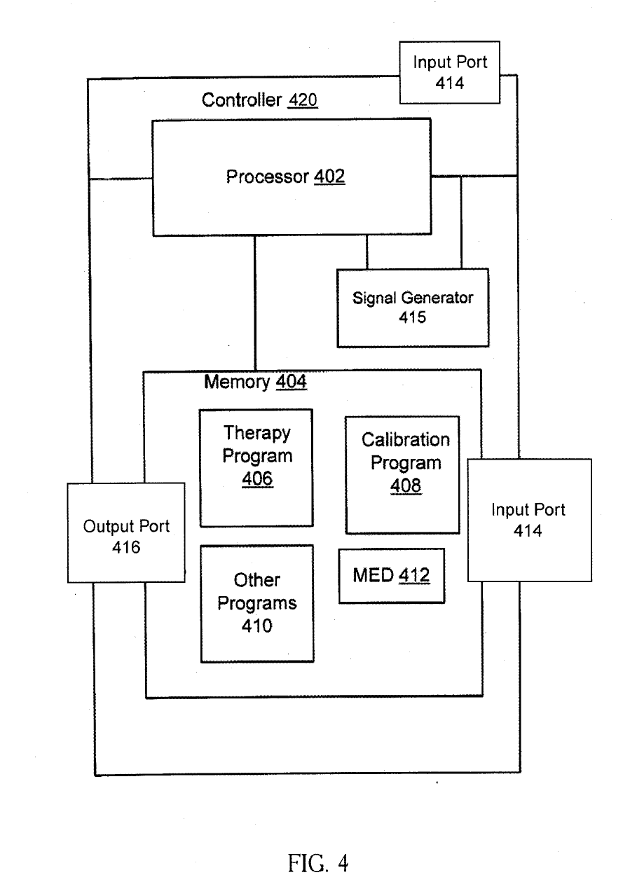

FIG. 4 shows a block diagram of an example of controller 420. Controller 420 may include processor 402, memory 404, and signal generator 415. Memory 404 may have a therapy program 406, calibration program 408, and/or other programs 410. Memory 404 may store MED 412 and/or other parameters such as the dose history previously applied to the patient. Controller 420 may also include one or more input ports 414 and one or more output ports 416. In other embodiments, controller 420 may not have all of the components associated with FIG. 4 and/or may have other components in addition to or instead of those associated with FIG. 4.

Processor 402 performs the therapy program and/or calibration programs referred to above and/or other programs. Memory 404 may include one or more machine-readable mediums that may store a variety of different types of information.

The term machine-readable medium is used to refer to any medium capable of carrying information that is readable by a machine, such as processor 402. One example of a machine-readable medium is a computer-readable medium. Although machine-readable medium of memory 404 is capable of storing information for a period of time that is longer than the time required for transferring information through memory 404, the term machine-readable medium may also include mediums that carry information while the information that is in transit from one location to another, such as copper wire and/or optical fiber.

Memory 404 stores programs that are executed by processor 402 and/or parameters used by those programs. In this specification, the word program is used to refer to any group of one or more instructions that cause a processor to perform at least part of a task when the one or more instructions are executed. In the example of FIG. 4, memory 404 may store therapy program 406 and/or calibration program 408 and or dose history program. Therapy program 406 and calibration program 408 include one or more instructions that cause processor 402 to perform the therapy and the calibration discussed in conjunction with FIGS. 1-3, respectively. Memory 404 may also store other programs 410, which are optional. If present, other programs 410 may include one or more other programs entered by the doctor or patient.

MED 412 (such as discussed in conjunction with FIG. 3) and/or other parameters may be entered by a patient or doctor and/or may be determined and/or stored automatically. One or more input ports 414 may be connected to one or more input devices for entering programs and/or parameters into memory 404. One or more input ports 414 may also receive input from one or more detectors used for calibrating radiation applicator, e.g. device 100. One or more input ports 414 may be useable as an interface to a computer or other machine that is used for programming controller 420. One or more input ports 414 may be useable for downloading programs, an MED, configuration parameters, and/or other information to controller 420. Input ports 414 may include an input port for a wireless signal (e.g., an antenna). Alternatively, a computer or other machine may be attached to one or more input ports 414, and used to either directly control radiation sources or control radiation source(s) via controller 420.

Signal generator 415 may produce a variety of different signals that vary in pulse width, pulse height, and/or pulse shape. Signal generator 415 may produce signals having different duty cycles based on the capabilities of power source 430, and based on how much heat is generated by radiation source(s) (e.g. radiation sources 102a-102n) while in an on state and/or a desired therapy. Signal generator 415 may be controlled by processor 402. Signal generator 415 is optional. In an embodiment in which signal generator 415 is not present, processor 402 may address radiation source(s) directly.

One or more output ports 416 may be associated with the controller 420 and may be connected, via electrical connections, to radiation source(s). There may be one output port 416 for each one of, or each group of, radiation source(s). One or more output ports 416 may be capable of being connected to one or more output devices, such as a monitor and/or display. By connecting an output device, it may be possible to view programs and/or parameters entered into memory 404 to aid in programming processor 402 and/or debugging one of the programs stored on memory 404. If signal generator 415 is present, some of the one or more output ports 416 may be connected to corresponding outputs of signal generator 415, and some of the one or more output ports 406 may be connected directly to processor 402 for communicating with an external device, such as a computer or terminal.

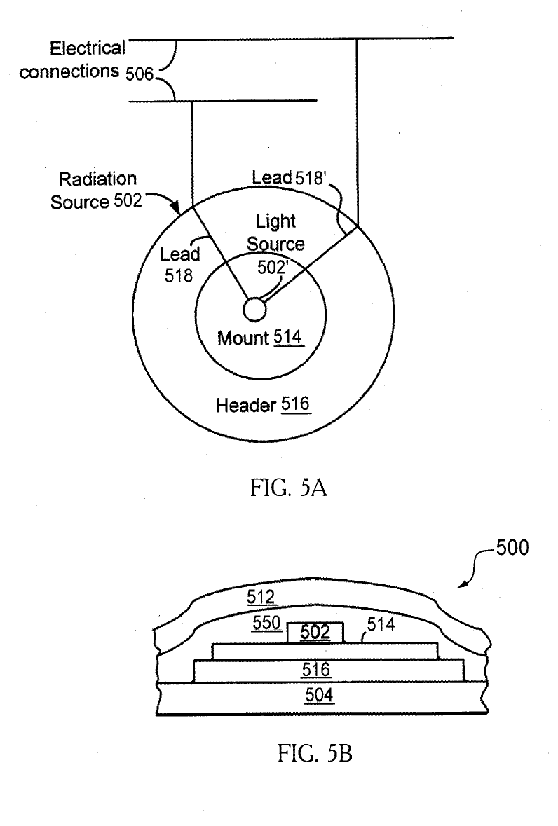

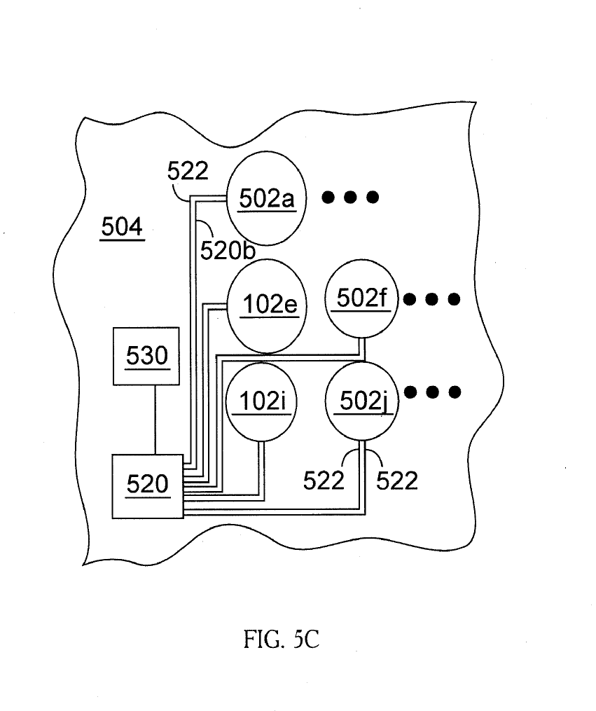

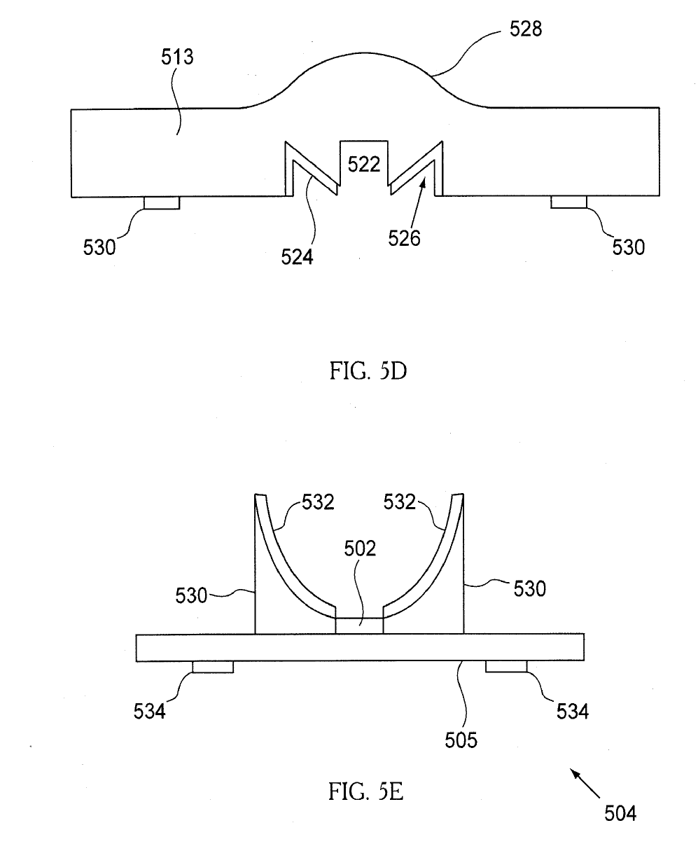

FIG. 5A shows a schematic diagram of an example of radiation source 500. Radiation source 500 may include the actual radiation source 502, such as a light source, and its supporting elements which allow the radiation source to function. For example, if the radiation source is a light emitting diode (LED), the supporting elements can include mount 514, header 516, lead 518, and lead 510; these supporting elements can be referred to as the radiation source module. In other embodiments, radiation source (or radiation source module) 500 may not have all of the components associated with FIG. 5A and/or may have other components in addition to, or instead of, those associated with FIG. 5A. Furthermore, as would be recognized by those skilled in the art, many variations of these basic components are possible. For example, the mount 514 could be made from any of many shapes, sizes, thicknesses, or from materials such as Beryllium Oxide (BeO), Aluminum Nitride (AlN), alumina, aluminum, copper, steel, MgF₂, or a semiconductor (e.g. silicon). The leads 518, 510 can be made from copper, silver, gold, alloys, or polymers as would be recognized by those skilled in the art. Header 516 can be made from a variety of materials or made into many shapes. Header 516 can also contain features necessary for heat transfer such as fins or dimples to increase the surface area of the header. The header can also be manufactured by depositing or molding metal (e.g. Kovar®, an alloy of iron, nickel and/or cobalt which has similar thermal expansion properties to glass, Westinghouse Electric & Manufacturing, Pittsburgh Pa.) directly onto a flexible material (e.g. silicone), which is part of the applicator 104 in FIG. 1. The radiation source can then be placed, using a die bonder, onto the deposited Kovar, after which wire bonds or soldered welds can be used to attach the radiation sources to a power circuit. Alternatively, the wire bonds can also be deposited on the flexible substrate (e.g. surface 104 in FIG. 1) using deposition processes such as electrodeposition, chemical vapor deposition, or physical vapor deposition.

Radiation source 502 may be a surface mount LED, or LED die, such as a UV LED die, blue light LED die, white light surface mount (SMD), Infrared (IR) LED or SMD, or UV LED SMD. As another example, radiation source 502 may be a small light bulb, resistive heater, or a device for generating microwaves, radiofrequency energy, X-rays, and/or radio frequency light. More specifically, radiation source 502 can emit energy in the immunosuppressive or anti-infective range of the ultraviolet spectrum. Wavelengths included in the immunosuppressive range of the ultraviolet spectrum include those from 295 nm to 320 nm and/or from 340 nm to 400 nm. In other embodiments where it is desired to treat infectious agents, radiation source 502 can emit ultraviolet light in the range 250-300 nm.

In an embodiment where radiation source 502 is a light source, mount 514 may hold light source 502 in place. Mount 514 may include a heat sink, circuit board, or a circuit board on top of a heat sink (e.g., a passive heat sink to diffuse heat over a larger surface area or an active sink to electrically pump heat away from the light generating regions). One example of a circuit board (sub-mount) is a gold-patterned ceramic such as beryllium-oxide (BeO) or aluminum nitride (AlN); the ceramic can act as a heat sink or a highly conducting heat transfer element through which heat conducts to the heat sink. Mount 514 may be a material such as Kovar alloy, which can act as a heat sink in addition to the ceramic material and is a very good material to bond beryllium oxide or aluminum nitride to because it (Kovar alloy) has a very similar coefficient of heat expansion. If mount 514 includes a heat sink, mount 514 may reduce the likelihood of light source 502 overheating and/or may otherwise extend the lifetime of light source 502 so that light source 502 lasts longer with a higher optical output per electrical input (efficiency) than if there were there no heat sink. Although in the example of FIG. 5, there is only one light source 502 on mount 514, there may be plurality of light sources on each mount 514. Light source 502 (e.g., an individual or multitude of UV LEDs) may be attached (e.g., bonded) to mount 514 using a eutectic metal or a solder such as gold-tin, lead-tin, other applicable eutectic solder material. Optionally, mount 514 may be textured (e.g., roughened) for scattering light or polished for specularly reflecting light. Mount 514 may be shaped for concentrating, diffusing, collimating, or dispersing light from light sources 502. Mount 514 may be flat, concave, or convex. If mount 514 is concave or convex, mount 514 may be elliptical, spherical, or hyperbolic, for example. Mount 514 may be composed of, or coated with, a reflecting metal such as aluminum or aluminum derivative. Mount 514 can additionally contain three-dimensional features 530 which are deposited on mount 514 (FIG. 5E).

Further, with respect to FIG. 5E a radiation source is depicted in the center of two three-dimensional pillars 534. The pillars can be deposited onto mount 505 or they can be attached after being made by another mechanism. Typical attachment processes can include a press fit, eutectic mount, adhesive mounting, ultrasonic welding, and light based curing. Mounting elements 530 can be electrical mounts, a material solely intended for the mounting process, a material to facilitate heat transfer, or a combination thereof The radiation source (e.g. a light source) can be placed in between the three-dimensional pillars 534 so that the radiation will reflect or refract forward from the three-dimensional pillars 534 in a pre-determined pattern outward to the body surface. As will be appreciate by those of skill in the art, the three-dimensional pillars 534 can assume any of a variety of configurations other than the pillars depicted without departing from the scope of the invention.

An advantage of placing pillars 534 around the radiation source or multiple individual radiation sources is that the radiation from the individual radiation sources can be captured independently from other radiation sources nearby. Such an arrangement can optimize light extraction and can direct the radiation in specific directions. Three-dimensional pillars 534 can be deposited on the surface 505 of the mount using processes such as eletrodeposition, chemical vapor deposition, physical vapor deposition, micromolding, electroforming, or other deposition processes known to those skilled in the art. In one example, mount 514 is made from a ceramic such as Beryllium Oxide or Aluminum Nitride. Standard physical vapor deposition processes can be used to then deposit conducting metallic layers such as gold or a eutectic metal such as gold-tin on the ceramic. With a conducting surface such as gold deposited on the ceramic, additional features can then be deposited (e.g. with an electrodeposition process) on the conducting metal which would reflect, focus, concentrate, disperse, or otherwise condition light. In another example, three dimensional features are not deposited directly but are produced in separate molds which are then applied to the surface 505 of the mount 514. When the surface pattern in the mount 514 is made from a eutectic metal, the mold placed on the mount surface and heat is then applied to the mount 514. The heat can weld the eutectic metal to the three-dimensional piece in the mold; after cooling, the mold is removed, leaving the mount 514 with a three-dimensional feature 530 welded to it. A combination of these processes can also be used in which three-dimensional features 534 are fabricated and then additional layers 532 are deposited on top of the three-dimensional features. For example, UV reflecting aluminum could be deposited on top of the three-dimensional features 534 on the mount 514. Light is then directed from radiation source 502 using one or all of these processes and/or structures.

Header 516 may protect light source 502 and mount 514 from being separated. Although in the example of FIG. 5A-E, header 516 has only one mount 514, there may be plurality of mounts 514 and each mount may have only one light source or may have a plurality of light sources. Similar to mount 514, header 516 may be shaped for concentrating, diffusing, collimating, dispersing, or otherwise reflecting light (e.g., with an aluminum reflecting layer) light from light sources 502. Header 516 may be flat, concave, or convex. If header 516 is concave or convex, header 516 may be elliptical, spherical, or hyperbolic, for example. Alternatively, there may be another optical component in addition to, or instead of, shaping and/or texturing mount 514 and/or packaging header 516 to have particular optical properties. Specifically, this additional optical component may be shaped for concentrating, diffusing, collimating, or dispersing light from light sources 502. The additional optical component may be flat, concave (for dispersing the radiation), or convex for concentrating the radiation. If the additional optical component is concave or convex, the additional optical component may be elliptical, spherical, or hyperbolic, for example. Header 516 can also contain three-dimensional microfabricated components as described above in the mount. The same or similar processes can be employed for the header.

In an embodiment, mount 514 and header 516 are separate components that are attached to one another. In another embodiment, mount 514 and header 516 may be two parts of the same component and/or only one of mount 514 and header 516 are used. If there is more than one light source on each mount 514 and/or within each header 516, the light sources may all have the same spectrum and/or may be associated with the same peak wavelength. Alternatively, there may be different light sources having different spectrums and/or peak wavelengths that are located on the same mount 514 and/or one the same header 516.

The leads 518, 510 supply power to light source 502 for activating light source 502 and keeping light source 502 lit. Further, leads 518, 510 may be connected to larger leads on substrate 104 that bring electricity to radiation source 502 (e.g., leads 518 and 510 may be connected to electrical connections 322). As will be appreciated by those skilled in the art, leads 510, 518 may be made from an alloyed, eutectic or non-alloyed, metal placed on or bonded to mount 514. Thus, current from power source 330 flows to controller 320, through electrical connections 322, and to one or more of radiation source(s) 102 (e.g., to leads 518 and 510, and then to light source 502, such as an UV LED), resulting in light, such as UV light, being output and subsequently biologic effect.

FIG. 5B shows a cross-section of an embodiment of radiation applicator 500. The embodiment of FIG. 5B includes flexible substrate 104, light source 502, mount 514, header 516, spectral conditioner 550, and optional patient interface 512. In other embodiments, radiation applicator 100 may not have all of the components associated with FIG. 5B and/or may have other components in addition to, or instead of, those associated with FIG. 5B.

Substrate 104 is discussed above in conjunction with FIG. 1 and elsewhere. Light source 502, mount 514, and header 516 are discussed above in conjunction with FIG. 5. Spectral conditioner 550 covers and may protect light source 502 from damage and/or may condition the radiation in one or more ways before it reaches the lesion. Spectral conditioner 550 may be one continuous layer of material that extends over all of region 506 or over all of substrate 504. Alternatively, spectral conditioner 550 may be a collection of patches of material, where each patch conditions the radiation from at least one light source, such as light source 502. In this embodiment, when the spectral conditioner 550 is a patch and individually covers one light source, the entire light source, including the covering 513, header 516, and mount 514 can be individually removed from the material 504 and then replaced on material 504. Depending on the embodiment, spectral conditioner 550 may cover a larger area than light source 502 but smaller than or equal to mount 514, cover a larger area than mount 514 but smaller than or equal to header 516, or cover a larger area than header 516 but not large enough to reach a covering of an adjacent radiation source.

Spectral conditioner 550 may make radiation applicator 500 more comfortable to wear, because the surface of spectral conditioner 550 that contacts the body portion can be smoother than the surface of radiation applicator 100 than if spectral conditioner 550 were not present. Spectral conditioner 550 and substrate 504 may form two layers of material, with light sources 502 sandwiched in between. Spectral conditioner 550 may be a layer of material, which may be transparent or translucent (e.g. to ultraviolet light between 250 nm and 320 nm), while a substrate 504 may be transparent, opaque, translucent, or reflective. If substrate 504 is reflective, substrate 504 may be specularly reflective or may scatter light. By making substrate 504 reflective, the efficiency of radiation applicator 500 is improved as compared to where substrate 504 is not reflective. By making either or both of substrate 504 and covering 513 a light scattering material, the uniformity of the irradiation may be improved as compared to if substrate 504 and/or spectral conditioner 550 do not scatter light. Spectral conditioner 550 may be made to scatter light using any of the structures discussed above in conjunction with the discussion of substrate 204 of FIG. 2. Spectral conditioner 550 may reduce efficiency (depending upon how much radiation it absorbs or otherwise prevents for reaching the patient), but may improve the uniformity of the irradiation and/or comfort to the patient.

Optional patient interface 512 may be an adhesive to help radiation applicator 500 adhere to the body portion being treated. Optional patient interface 512 may be a layer of adhesive material (e.g., glue) that partially or completely covers one surface of radiation applicator 500, such as covering 513. Optional adhesive may be included in an embodiment in which radiation applicator 500 is a bandage that sticks to a portion of skin of a patient, for example. Optional adhesive may the adhesive discussed in conjunction with FIG. 1 and/or substrate 504. In addition to glue, patient interface 512 may incorporate therapeutic substances designed to prevent damage and/or enhance the therapeutic efficacy of the radiation delivered by radiation applicator 500. Examples of potentially protective compounds include titanium oxide, zinc oxide, and others well-known to those skilled in the art. Examples of compounds to improve efficacy can include photosensitizers such as the broad categories of psoralens, the porphyrin family, and other photosensitizers which are well-known in the art. FIG. 5C shows a bock diagram of an example of an embodiment of radiation applicator 500. FIG. 5C includes radiations sources 502a, 502b, 502e, 502f, 502i, and 502j, substrate 504, controller 520, power source 530, and electrical connections 522 (such as 522a-522t). In other embodiments, radiation applicator 500 may not have all of the components associated with FIG. 5C and/or may have other components in addition to or instead of those associated with FIG. 5C.

Radiation source(s) 502a, 502e, 502f, 502i, and 502j are specific ones of, or specific groups of, radiation source(s) 502 (e.g. 502a-502n), which are discussed in conjunction with FIGS. 1 and 5A shown in FIG. 5C. The sets of three dots after radiation source(s) 502a, 502f, and 502j represent any number of radiations sources. Although pairs of letters, such as “e” and “f,” and “i” and “j,” may represent pairs of consecutive numbers that are smaller than the number represented by “n,” there may be any number of radiation source(s) between radiation source(s) 502b and 502e, between radiation source(s) 502f and 502i, and between radiation source(s) 502j and 502n. Substrate 504 is discussed in conjunction with FIGS. 1 and 5B and elsewhere. Controller 520 and power source 530 are discussed in conjunction with FIG. 3 and elsewhere.

Electrical connections 522 (e.g. 522a-522t) are paired with one another. Each pair completes a circuit between controller 520 and one of radiation source(s) 502 (e.g. 502a-502n). The pattern of electrical connections 522a-522n is different than electrical connections 322 (FIG. 3). In this embodiment, each radiation source or group of radiation source(s) has its own ground or return electrode and can be controlled independently by controller 520.

Turning now to FIG. 5D, a close-up of a molded covering 513 with optical components built-in is depicted. In this embodiment, covering 513 is placed over the radiation source which then resides in space 522. The covering 513 can be a molded piece, a machined piece, a lithographically formed piece, or a combination of these. Angled indent 526 represents a three-dimensional component of the piece (covering) which is a planned feature of the molded piece. Layer 524 is an optional layer which can be deposited on the angled indent 526. Layer 524 can be reflective, refractive, absorbing, or diffusing, having a different index of refraction from the covering 513 material. Diffuser 528 is another feature which can optionally be built into the molded covering 513. Diffuser 528 is a feature adapted and configured to further direct, focus, diffuse, or otherwise condition the radiation leaving source 502. One or more projections 530 can be deposited or glued onto covering 513. These projections 530 can be adapted and configured to enhance heat transfer, enhance bonding, or enhance conduction to an underlying mount. Although covering 513 depicts space for only one set of radiation sources 522, those skilled in the art will recognize that more than one radiation source or sources can be included in covering 513.

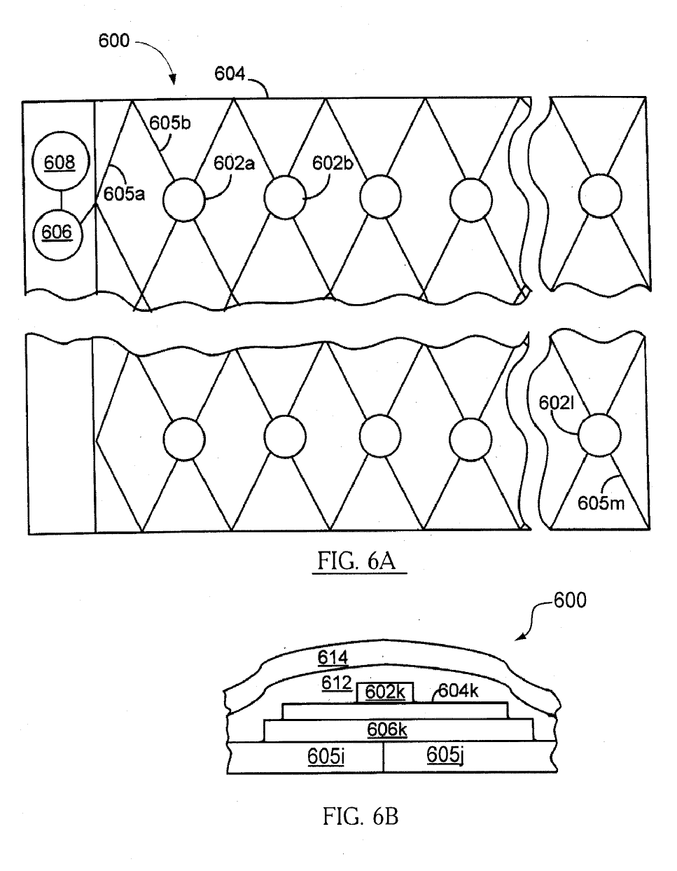

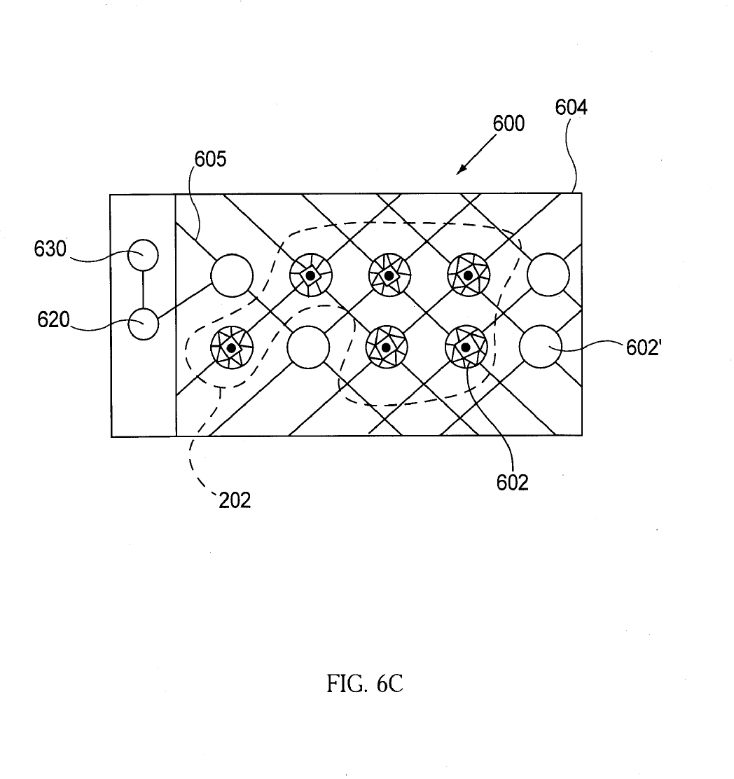

FIG. 6A shows a radiation applicator 600. Radiation applicator 600 includes radiation source(s) 602a-l, substrate 604 having cords 605a-605m, controller 606, and power source 608. In other embodiments, radiation applicator 600 may not have all of the components in FIG. 6A or may have other components in addition to, or instead of, those in FIG. 6A.

Radiation applicator 600 may be an embodiment of a radiation applicator. Radiation source(s) 602a-l could be of any of the types of radiation source(s) as radiation source(s) 602 (e.g. 602a-602n). Substrate 604 may be a mesh (e.g., a flexible net) that is made of crisscrossing cords 605a-m, which may be an embodiment of substrate 104 in FIG. 1. For example, the flexible net that makes up substrate 604 may be a bandage which is highly elastic. Radiation source(s) 602a-l can be placed at the intersection of individual cords 605a-m of substrate 604. In an alternative embodiment, radiation source(s) 602a-602l may be placed on other parts of cords 605a-605m in addition to, or instead of, being placed at the intersections of two of cords 605a-605m. Controller 620 may be the same as controller 320 of FIG. 3 and power source 630 may be the same as power source 330. Cords 605a-m may carry or may include electrical connections 622 and/or optical fibers that bring electricity and/or optical communications from controller 606 to radiation source(s) 602 for powering and/or communicating with radiations sources 602a-6021. The configuration of cords 605a-605m allow radiation source(s) 602a-602l to cool by allowing air to pass across the backs of radiation source(s) 602a-602l. The configuration further allows for flexible spacing between the intersections of the cords. In this way, the material (the nodes) can be spread apart by applying force to the edges of the radiation applicator 600 and then allowed to return to the prior spacing when the edges are allowed to return their previous spacing. Although the embodiment of FIG. 6A does not include a region such as region 106, in an alternative embodiment, substrate 604 may include a region 606.

FIG. 6B shows a cross-section of an example of an embodiment of a radiation applicator 600. The embodiment of FIG. 6B includes light source 602k, mount 604k, cord 605i, cord 605j, header 606k, spectral conditioner 612, and optional patient interface 614. In other embodiments, radiation applicator 600 may not have all of the components associated with FIG. 6B and/or may have other components in addition to, or instead of, those associated with FIG. 6B.

Light source 602k, mount 604k, and header 606kare the light source, mount, and header of one of radiations sources 602a-602n. Light source 602k, mount 604k, and header 606kmay be embodiments of light source 602, mount 614, and header 616, respectively. Similarly, spectral conditioner 612 and optional patient interface 614, which may include adhesive, may be an embodiment of spectral conditioner 650 and optional patient interface 612, respectively. Cords 605i and 605j are two of cords 605a-605l. Cords 605i and 605j are a pair of cords that criss-cross one another under mount 604k.

As discussed above, the radiation applicator 600 can be adapted to be placed on a patient at a target body surface such that it covers, or substantially covers, a therapeutic surface area. As shown in FIG. 6C, the radiation applicator 600 is applied to the target body surface such that the radiation applicator 600 covers a lesion 20, to which therapy will be delivered. Further radiation sources 602, 602′ associated with the radiation applicator 600 can be selectively activated such that a first subset of radiation sources (602) is on, while the remainder of the radiation sources (602′) are not on. As illustrated, the first subset of radiation sources 602 are positioned within the radiation applicator 600 such that the radiation sources 602 can apply therapy to the lesion 202. As will be appreciated by those skilled in the art, the first set of radiation sources 602 can be further divided into subsets that are separately programmable to deliver different therapeutic doses. This embodiment would be appropriate where, for example, a lesion to be treated has, within the lesion, areas that require more therapeutic treatment than other areas (e.g., a border region of a lesion might require less therapy, than a central portion). Radiation applicator 600 may also in-part comprise detectors that can sense certain physical attributes of a body surface that may differentiate a therapeutic region and a non-therapeutic region. The detectors, for example, can define the region of a lesion such that radiation sources covering the lesion region will be activated while those not covering the lesion region will not be activated. The detectors may detect one or more parameters including, but not limited to: temperature, electrical impedance, photoreflectance, thickness, hardness, moisture, and acoustic reflections. Where the detectors measure photoreflectance, measurements may include one or more of the following: roughness, color, and fluorescence.

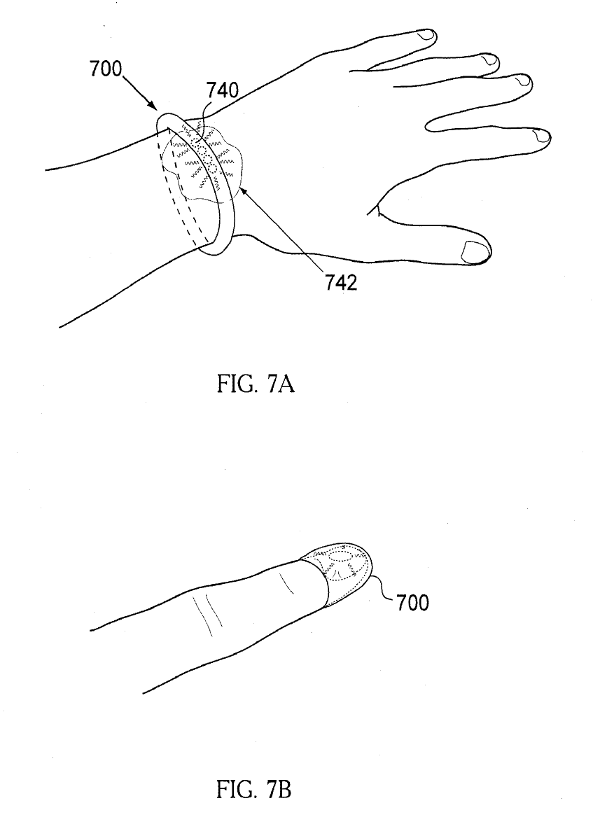

FIG. 7A shows an embodiment of the therapeutic device 700 in which radiation sources are incorporated into a device which can be worn or otherwise fixtured, carried, or attached to a patient while the therapy to treat a skin disorder is being applied. Although the device 700 of the embodiment illustrated in FIG. 7B has the form of a bracelet, the radiation sources 740 can be incorporated into any material which can at least partially cover or are in direct or indirect contact with the patient's skin 742. For example, the therapeutic device 700 may have the form of a bandage, blanket, any articles of clothing, a ring, jewelry, a hat, a wristband, a shirt, a sock, underwear, a scarf, a headband, a patch, a gauze pad, or any other wearable article, etc. The device 700 may be adapted and configured to communicate with photodetectors, which can continuously readjust the device's output or can be configured to detect a disease state of the skin so that the optical therapy can be applied.

In another embodiment, several devices 700, 100 (e.g., bandages) are brought together or applied to treat a larger area. In one embodiment, a kit having different sized bandages is provided. Adhesive can be a component of the kit and/or a component of the bandages. The individual sized bandages can be fit together to irradiate different shaped and sized areas or lesions. With such a “wearable” device 700, a patient can treat his or her disorder (e.g., psoriasis) while performing other tasks or sleeping and can treat small or large areas of disease in a time- and cost-effective manner.

Such a localized therapy is also safer than treatments which apply light over a broad area of skin because portions of the skin which are not psoriatic can be unnecessarily exposed to ultraviolet light. With the LED systems described above, broad-band or narrow-band optical therapy can easily be applied to the skin depending upon clinical requirements. In addition, photodetectors may be integrated into the therapeutic device 700 for feedback control of the therapy. Internal body cavities can be treated as well with permanent or semi-permanent optical therapy devices 700. For example, in one embodiment, inner ear infections are treated by placing an optical therapy device 700 inside or proximal to the ear canal.

FIG. 7B illustrates an optical therapy device 700 used to treat fungal infection of the nail beds. In such a case, tinea infections of the nails may be treated with the device by choosing appropriate optical wavelengths (e.g., 255-320 nm) for the radiation sources. The optical therapy device 700 has the form of a bandage or Band-aid®. Such a device 700 allows patients to go about their daily lives while the treatment is being applied. The device 700 is constructed using the principles and methods described above. Device 700 can be used in combination with photosensitizers or photodynamic agents to better treat the nailbed. In another embodiment, the device shown in FIG. 7B is used to treat nail psoriasis in which case wavelengths between 295 nm and 320 nm, typically would be used.

The devices and radiation source(s) disclosed herein can be used for therapies such as psoriasis or other skin disorders currently treated with radiation (e.g., vitiligo, cutaneous T cell lymphoma, fungal infections, etc.). The preferred action spectrum to treat psoriasis is approximately 308-311 nm. In addition, narrow-band radiation is generally more effective than broad-band radiation. One limiting factor in current modalities and technologies for the treatment of psoriatic lesions is that typical devices available on the market today are large and expensive, and generally require patients to visit a physician's office for treatment. Home-treatment devices are typically large fluorescent lamps that are adapted to treat a broad area rather than a localized region. Whether in the home or in the office of the medical practitioner, the therapy takes time out of the patient's daily schedule. In addition, it is typically difficult for a patient to perform other tasks while the therapy is being applied. Furthermore, with current technology, it is difficult to treat a small area of the skin with narrowband light. Lasers are sometimes used to do so, but lasers are generally expensive and are not practical as home-based therapy devices.

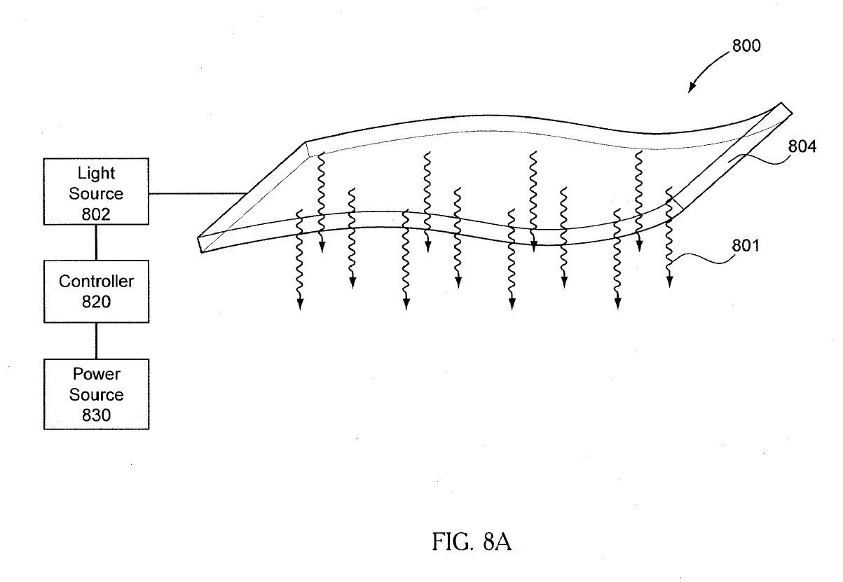

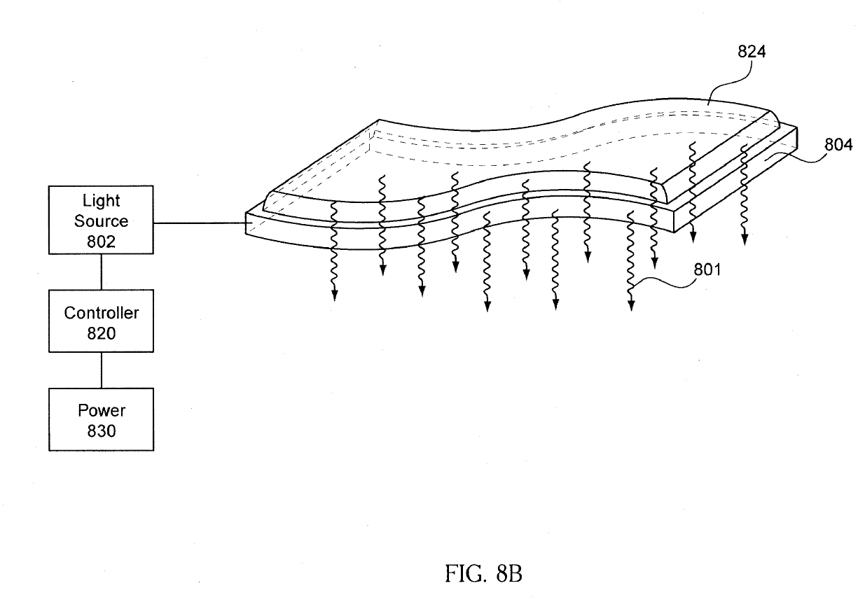

As will be appreciated by those skilled in the art, one challenge of providing uniform illumination to a target body surface is the high degree of varying curvature of the surface from location to location on a body. For example, a uniform approximately planar light source incident upon a flat surface will provide a uniform intensity distribution across that surface. However, intensity distribution from the same planar source incident upon a curved surface can vary greatly as the curved surface provides in effect a variable degree of distance from the light source. As depicted in FIG. 8A a light emitting device 800 is adapted to provide phototherapy to a patient's body. The light emitting device 800 is a therapeutic treatment apparatus that is adapted and configured to conform to a target area of a patient's body. The light emitting device 800 is further comprised of a light delivery element 804, such as a substrate, that is adapted and configured to deliver light and that that is flexible and generally conformable to a target body surface. The light 801 transmitted by this device 800 is such that its near field optical intensity is substantially uniformly distributed over the regions emitting light. The near field optical intensity is here described as the light intensity that is close to the exit plane a given optical element. Qualitatively, close in the preceding sentence is defined as a distance which is small as compared to the size of the optical element and is concurrently described as the a distance over which the light intensity does not substantially diminish. Therefore, a device 800 with these two features provides a uniform phototherapeutic treatment to virtually any target body surface. Additionally, the light delivery element 804 should provide a high degree of wearability for a user at a plurality of locations on the user's body and the physical dimensions of the light delivery element 804 are ideally ‘low profile’ such that a thickness of the device 800 is small compared to its length and width dimensions. Additionally, the flexibility of the light delivery element 804 facilitates application of the device to a patient while the patient is in motion (e,g., performing routine daily activities) as well as delivery of therapy to a patient while the patient is in motion.

In the embodiment depicted in FIG. 8A, any suitable light source 802 can be employed, for example, one or more light emitting diodes (LEDs), an arc lamp, or one or more laser diodes. The light source 802 can be adapted and configured to direct light to the light delivery element 804 which is operated by a controller element 820 and powered by a power supply 830. The actual dimensions of a device 800 can be varied within the scope of the invention and may be dependent upon the particular target body surface to be subjected to phototherapy as well as to any portability issues. Additionally, the length, width and depth of a light delivery element 804 can be varied and may be further dependent upon the particular target body surface to be subjected to phototherapy. For example, a device 800 used to treat an area of the torso is likely to be comparatively larger than a device to treat an elbow. The light source 802 emits light 801 at a specific therapeutic wavelength or alternatively within a range of therapeutic wavelengths to include visible (400-800 nm), infrared (800-2000 nm), ultraviolet (200-400 nm) light as is suitable for a particular treatment regimen. For example, ultraviolet light is used to treat in the wavelength range of 300-320 nm (commonly referred to as the ultraviolet B range) is typically used for the treatment of psoriasis. In a typical modern phototherapy treatment, light with a specific wavelength of 311 nm is used; this is often referred to as narrow band ultraviolet B therapy.

In another embodiment, depicted in FIG. 8B, the light delivery element 804 is directly coupled to a heat absorbing structure 824 that is typically located on an opposite side to the side that transmits the treatment light. The heat absorbing structure 824 is designed such that waste heat created by the delivery element can be dissipated directly on the device and therefore substantially allows the light delivery element to remain at or below typical body surface temperature. However, it may also be configured such that a small amount of heat, such as a therapeutic amount, is applied to the target body surface, while the remaining heat generated is dissipated. In an embodiment, the heat absorbing structure 824 may be comprised of a bladder 825 that is filled with a solid, liquid, gas, or mixture thereof that has suitable heat capacity. For example, 5 watts of power may be consumed by the delivery device during 10 minutes of operation thus creating approximately 3000 Joules of heat. To sustain a temperature rise from ambient (25 C) to body temperature (37 C), it would be suitable for the bladder to contain at least 63 grams of water. Additionally, such a bladder may be temporarily removable such that it may be externally heated or cooled prior to device operation to then subsequently be affixed in the heat absorbing structure to provide enhanced heat capacity based upon device use. In the embodiment depicted in FIG. 8B, any suitable light source 802 which could be one or more LEDs, an arc lamp, or one or more laser diodes, is directed to the patient interface element 812 and is operated by a controller element 820 and a power supply 830.

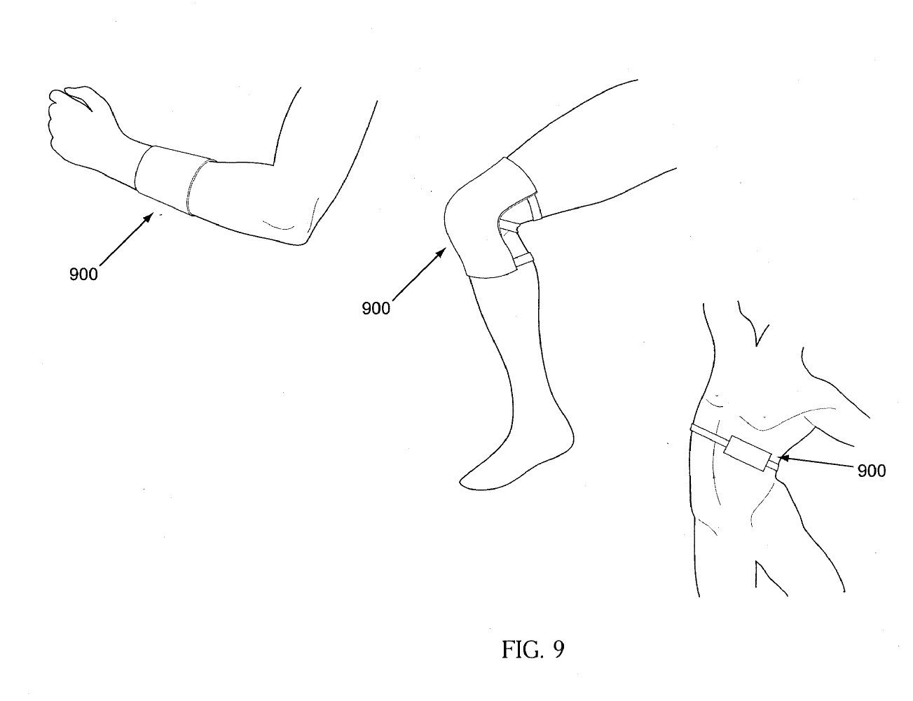

FIGS. 9A-C depicts several potential examples of typical use of the described medical device. An essential feature of this device 900 is that it is wearable. In all examples of FIG. 9, the light delivery element conforms to the area of treatment and allows for unencumbered activity by the user. In FIG. 9B, the device is shown providing therapy over a high curvature, moving joint such as the knee. The device is also intended for other high curvature yet static body locations such as a forearm as shown in FIG. 9A. Additionally, the device 900 can be configured to treat large areas of relatively lower curvature surfaces such as the front torso, as depicted in FIG. 9C. However, as will be appreciated by those skilled in the art, the device can be configured to, for example, be applied to the lower back, as well as other body surfaces. The described device 900 can be affixed to a particular area of the body by any suitable attachment mechanism, such as an adhesive film, one or more straps, a material over wrap, or a cuff. An effective application of such attachment mechanism may in turn facilitate mobility of the patient during treatment thus rendering the entire device to be wearable. It is understood that a comfortable therapeutic device could be used on most parts of the human anatomy. It will be appreciated by those skilled in the art, that embodiments of the invention can potentially be applied to any part or surface of the body. For example, a patient with psoriasis may have areas of the disease on areas of the body, such as a limb, that have an inherent high degree of surface curvature.

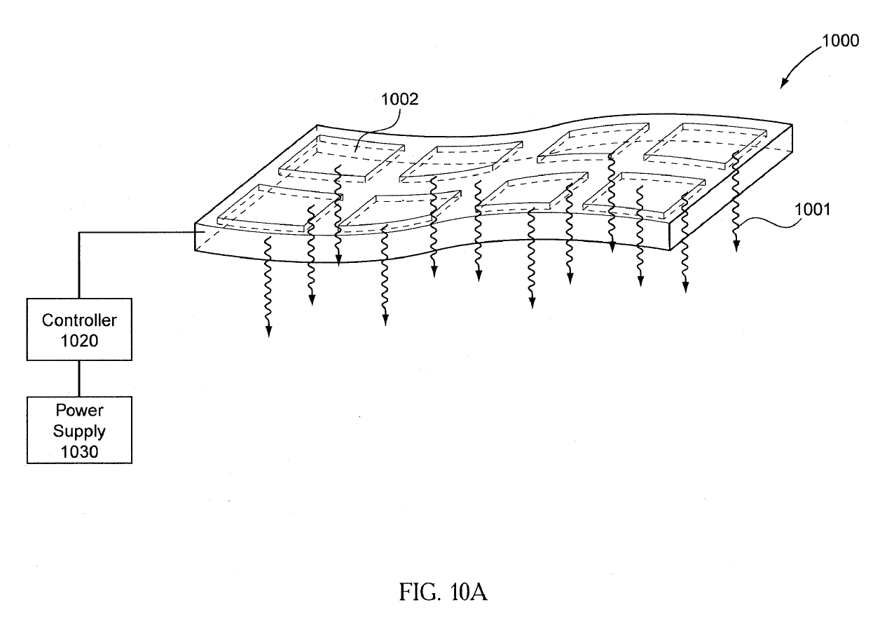

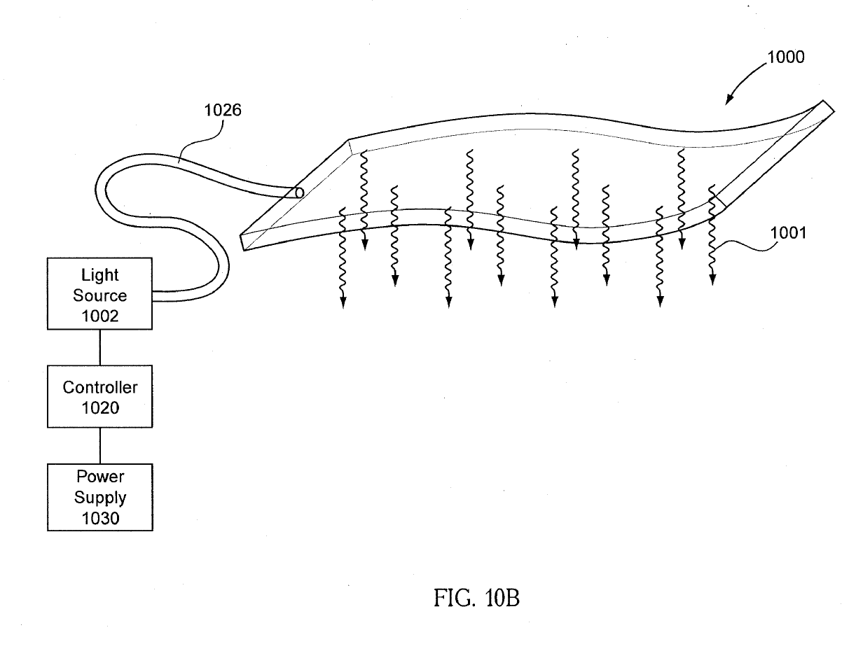

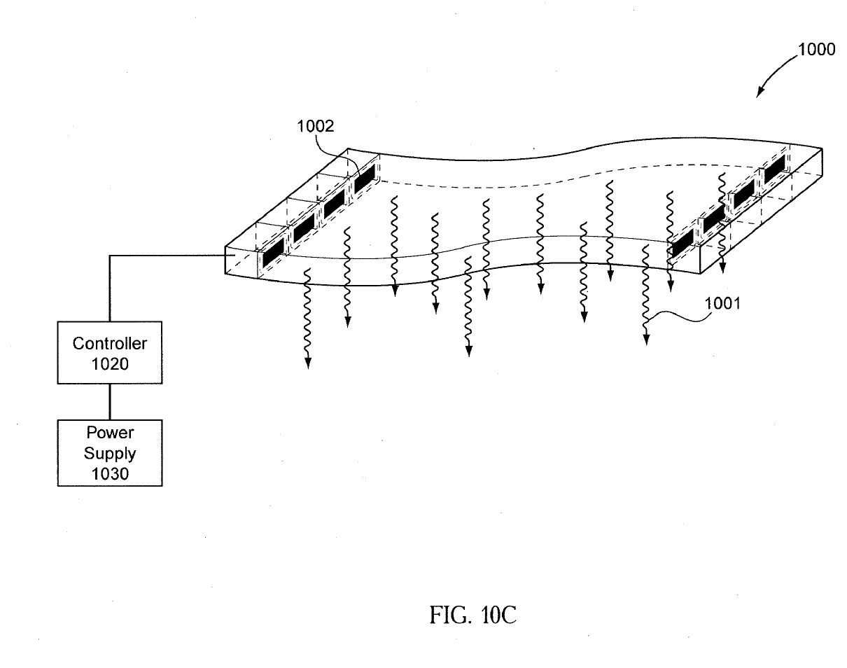

FIG. 10A describes an embodiment of a flexible, conformal device 1000 with light sources 1002 are incorporated into a conforming substrate. The flexible, conformal device 1000 is yet another embodiment of a therapeutic treatment apparatus. The light sources 1002 are aligned to provide light 1001 in the direction of transmission of the delivery element, ostensibly in a direction towards the body surface. The light sources 1002 can be light emitting diodes, laser diodes, or any other light source commensurate or adaptable to the size, shape, and wearability of the delivery device. The arrangement of light sources is adapted to provide sufficient light over all areas of the portions of the device intended to transmit light to a body surface. In some specific embodiments linear and array-like configurations of light sources may be used to achieve the light dispersion objective. It is also possible to arrange the light sources in any other regular or non-regular pattern such as a circle, where the spacing between each light source may or may not be equivalent. These light sources are also operated by a controller element 1020 and a power source 1030. In this embodiment, each light source 1002 will have a light integrator associated with it that controls the light distribution onto the target surface.

FIG. 10B describes an embodiment of a flexible and conformal therapeutic light delivery device 1000 emitting therapeutic light 1001 with a fiber optic light guide 1026 as an input source from a fixed external light source 1002. In this embodiment, fibers from the fiber optic light guide 1026 terminate into light integrators which control the light distribution onto the target surface. As with other embodiments, the light source may be an arc lamp, a laser, a plurality of laser diodes, a plurality of LEDs, or any other suitable method of generating therapeutic light. As shown, the light source is operated by a controller element 1020 and a power supply 1030. In this embodiment, the light generation site is in a remote location as compared to the light delivery site. This may be advantageous in view of typical light source inefficiency. For example, as heat is generated along with light, it is generally desirable in the therapeutic setting to dissipate heat in and around the location of light generation. However, by decoupling the generation of light from the delivery of light, associated challenges of light delivery along with heat dissipation are also decoupled and can be addressed separately.

FIG. 10C illustrates a flexible light delivery device 1000 with one or more light sources 1002 incorporated into the device 1000 and aligned to provide light perpendicular to the direction of transmission of the delivery element to the target body surface. The light sources can be a plurality of light emitting diodes, a plurality of laser diodes, or any other light source commensurate or adaptable to the size, shape, and wearability of the delivery device. The light 1001 is distributed onto a target body surface using light integrators which operate on the optical principles of reflection or refraction or other suitable optical means. An analog of this delivery scheme is similar to the backlight of a typical LCD screen used in portable electronics. These light sources are operated by a controller element 1020 and a power source 1030. This embodiment effectively clusters the light sources in a minimum of distinct locations thus in turn minimizing the extent of electrical and mechanical connections necessary for their operation.

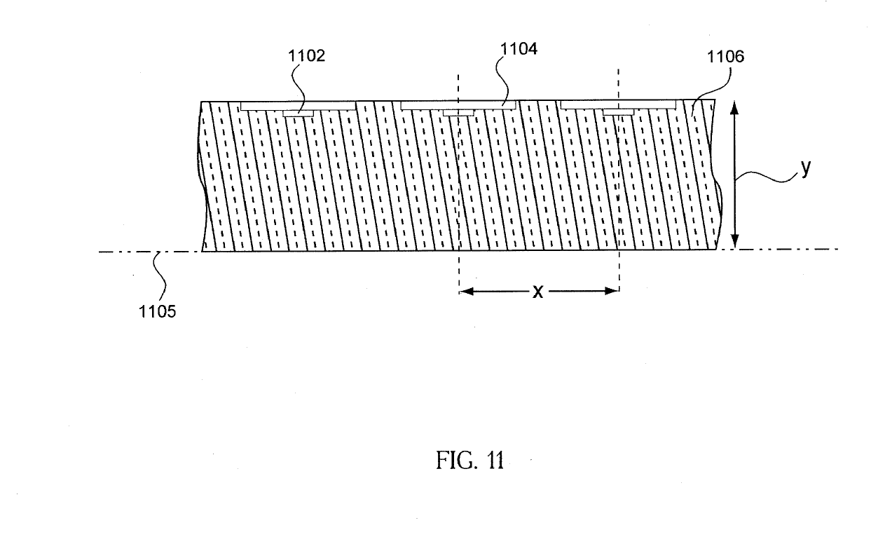

FIG. 11 depicts a cross section of an embodiment of a flexible, conformal light delivery element residing on a plane of contact 1105. Light sources 1102 and related housings 1104 are directly disposed on a continuous, optically transmissive material 1106. The distance between the light sources 1102 and the plane of contact 1105 is distance Y. The material pliancy is driven by both its thickness and its soft nature, such that it is both conformal and comfortable when applied to a body surface. For example, this material should not have a durometer value that exceeds 70 on the Shore A measurement scale; thus material is typically selected which has a durometer of less than or equal to Shore 70 A. Additionally, the material can be at least partially transmissive to therapeutic light. The light sources 1102 are spaced a distance X apart in a linear fashion. This concept can be extended to a two dimensional array with all light sources equidistant from one another, again at a spacing of X. As a design constraint to enable the delivery of uniformly distributed light, the spacing X in this embodiment is less than or equal to thickness Y. This relationship arises because each light source is considered to be a point source, or Lambertian, emitter.

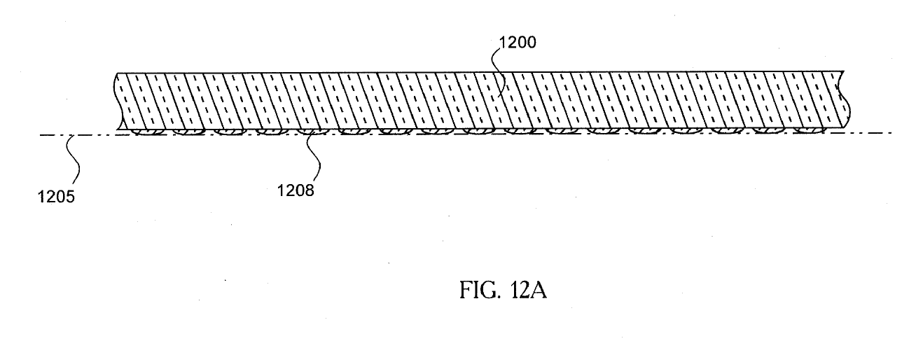

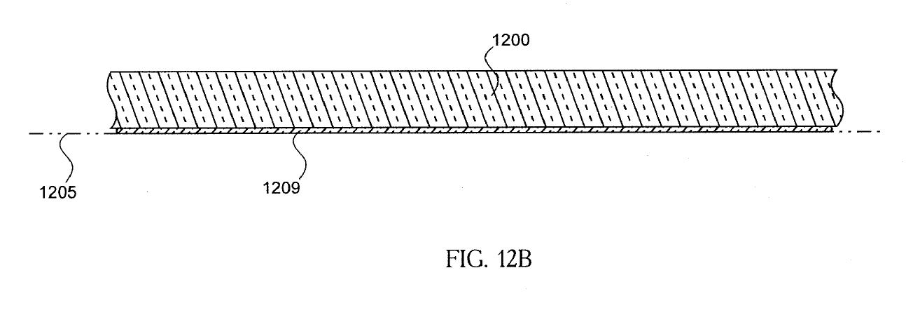

FIG. 12A illustrates a cross sectional view of an interfacial feature 1208 between the plane of contact 1205 of a patient and any conformal, flexible light delivery element 1200 described herein. This feature 1208 may be composed of the same substrate that comprises the light delivery element and is intended to not disrupt the optical output of the device yet concurrently provide less than 100% body surface contact between the device and the patient. This arrangement may be beneficial if, for example, the target body surface requires ventilation during the duration of a phototherapy treatment. As is depicted, hemispherical bump features, of comparably small thickness to that of the delivery device are located at regularly spaced intervals along the entire interfacial region. Although bumps are shown, alternative similar sized features such as ridges or rings may also be employed.

Another feature depicted in FIG. 12B is the intentional incorporation of an interfacial material 1209 between the plane of contact 1205 with a patient and the conformal, flexible light delivery element 1200. The local irregularity of patient's body surface may make it favorable to apply a material such as an optically transmissive gel or emollient to enhance the optical coupling between the emitting and receiving surfaces. Alternatively, a disposable film or thin sheet can be placed between the flexible substrate of the device and the target body surface. This may be beneficial to keep the therapeutic device clean of oils, exfoliate, or other material picked up with contact with the body surface. The disposable thin film is at least partially transmissive to the therapeutic wavelength as is emitted from the device.

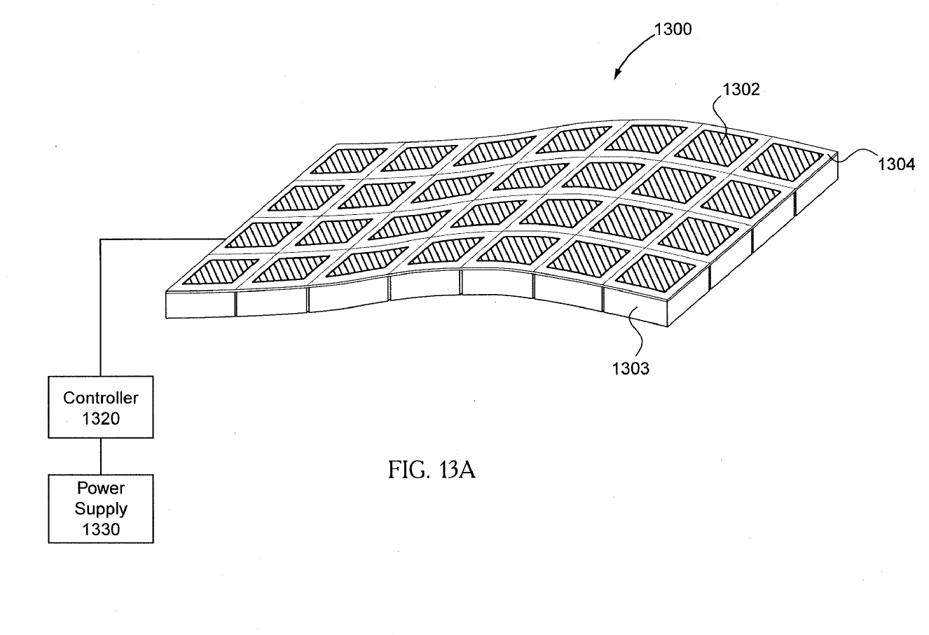

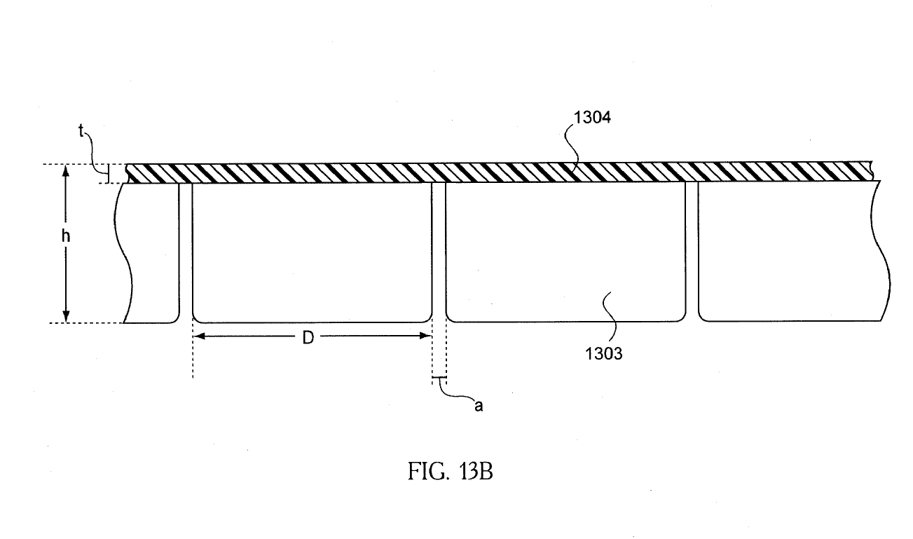

FIG. 13A depicts yet another embodiment of the described invention. A feature of this embodiment is the association of each light source with an individual, geometrically defined unit cell 1303. The unit cell functions on a defined scale to individually accomplish the task of the device as a whole; in acting substantially as a light integrator, each unit cell 1303 is itself transmitting and distributing light in a substantially uniform manner from an associated light source. Therefore the unit cells act as an ensemble to individually deliver substantially uniformly distributed light as parts of device that conforms to a patient body surface. The unit cells 1303 as shown are roughly cuboid in shape but may take other shapes as well. They are affixed as an ensemble to a unifying flexible substrate 1304. The flexible substrate may be a rubber material, including natural and synthetic rubbers, or may be fabric, cloth, thermoplastic, flexible metal, elastomer, or any combination thereof Alternatively, the flexible substrate can be formable such that once it is formed in a shape it will remain substantially in that shape until additional deformation is applied. Additionally, the light sources 1302 are operated by a controller 1320 and a power source 1330. In an embodiment, this controller 1320 is a programmable microprocessor and the power source 1330 is a battery and both are located either as a single component or as distinct separate components from the therapeutic device. In another embodiment, the controller and battery are integrally mounted to the therapeutic device 1300. In an embodiment, the unit cell 1303 is in part functioning as a waveguide.

FIG. 13B is a cross sectional schematic view of several unit cells 1303 (depicted without associated light sources) with a lateral dimension of D and a spacing between each unit cell of a. For purposes of achieving more uniform light distribution, D is typically larger than a. For example, D may be 1 cm while a may be 1 mm. A thin layer of flexible material 1304 which acts as a substrate for the ensemble of unit cell features may also be provided. The thickness t of this flexible layer 1304 is comparatively thin, e.g. 1 mm, when compared to the overall thickness h of the light delivery element, e.g. 1 cm. This provides the flexibility of the ensemble assembly. The flexible substrate material 1304 can be a material identical to that substantially comprising the unit cell, such as silicone rubber, or may be a separate material as has been previously mentioned. Additionally, the contact point of the flexible material and the unit cell can be at any location along height based on suitability for design. As shown, the cells are connected at the extreme edge of the top of the cell. However, they could be connected at the lower extreme edge, or at any location between the edges. In an embodiment, the unit cell is more rigid than the flexible substrate, based either entirely on thickness considerations or alternatively based upon materials selection. Therefore, in this embodiment, the ensemble device derives its flexibility and conformal nature by combining semi-rigid though defined unit cell blocks with a flexible substrate.

The unit cell 1303 serves several unique, enabling functions for light distribution. First, light sources 1302 employed in this particular application, such as LEDs, lasers, or fiber optically delivered light, can often be considered as point sources of light. Since the intensity of a point source decreases in a quadratic fashion with respect to the distance from the emitter, it is important that the distance from the emitter to the plane of contact be kept constant. In a semi-rigid unit cell configuration, each cell will retain its shape when placed in contact with a curved body surface; therefore, the intended uniform optical distribution will be retained despite use in a myriad of configurations and application to surfaces with varied curvature. For example, a single device could be applied to deliver a therapeutic treatment at different times to a knee, an elbow, a calf, and a region of the lower back without any substantial alteration to its form or functionality. Secondly, geometrically defining each unit cell by essentially giving it sidewall features enables the bulk of each unit cell to act as an optical integration unit. Internal reflection caused by at least a partially transmissive coating or other mechanism at the wall boundary can act to redistribute incident light as it is on the path towards the target plane of contact with the body surface. A method of reflection is known as total internal reflection, and is caused by a contrast in the index of refraction between the material comprising the light integrator and air. This reflection phenomenon at the unit cell wall boundary will can cause redistribution of transmitted light at the plane of contact of such a unit cell. These effects can advantageously compensate for the typical aforementioned spatial decrease in light intensity with respect to the distance from the light source. It is important to note that some internal reflection does not disallow some therapeutic light from exiting the light integrator element at any point along the sidewall feature. This characteristic can in fact enable light to reach areas of a body surface that are not in close contact with a light integrator element, such as the finite space in between unit cell structures.

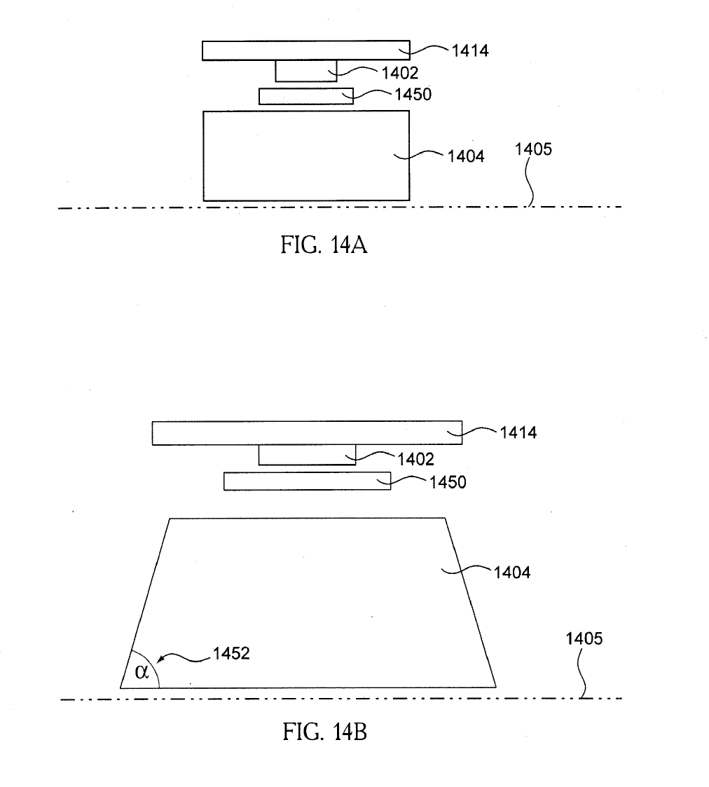

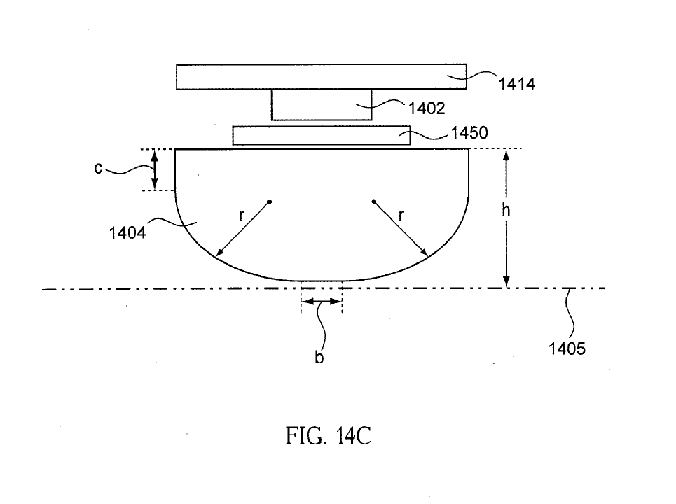

A generalized picture of the unit cell is shown in FIG. 14A. The unit cell is adapted and configured to form a therapeutic treatment apparatus. In this embodiment, the device or apparatus is comprised of three building blocks. The first building block is the light integration unit 1404 which typically is positioned on the plane of contact 1405 with a target portion of a body surface. A key material property necessary for this light integration unit is that it be at least partially optically transparent to the desired range of wavelengths used in the specific phototherapy treatment. Additionally, this material should be rigid or semi-rigid. The most desirable material for this application is an acrylic or a silicone elastomer, while epoxy, polycarbonate, fused silica, and combinations thereof may be used. Second, a light source 1402 and light source housing 1414 are described. As is shown, these features are located above the light integration unit. The light source housing is generalized to include any combination of relevant componentry such as device packaging materials and components, electrical contacts, a circuit board, or a flexible conductor. An optical element 1450 which functions to aid in the distribution of the light emitted from the light source 1402 may also be provided. This element 1450 can be a convex, concave, aspheric, diffractive, Fresnel type, or free form lens. It is also possible to incorporate this optical element 1450 directly into the light integration unit 1404 by molding or any other mechanism as well as it is possible to integrate this optical element 1450 directly into the light source housing 1414 feature. An alternative embodiment does not make use of a specific intermediary optical element.

Applications may dictate the formation of various prism shapes of the light integration unit that are not specifically a cuboid geometry with right angles. A cuboid is defined here as an elemental shape composed of six nearly rectangular sides. FIG. 14B is a cross sectional representation of an embodiment of a unit cell structure with light source 1402 and light source housing 1414, optical element 1450, and a light integration unit 1404. The light integration unit is typically positioned on the plane of contact 1405 with a body surface and has a roughly trapezoidal shape characterized by defining an angle of a of the lower corner vertices 1452. These unit cell shapes may be thus optimized to enhance distribution of transmitted light to a body surface. The angle a may be selected from a range between 5 and 90 degrees. Additionally, this angle may describe the lower corner vertices of other geometric shapes comprising the unit cell element such as a pyramid or a hexagonal prism.

FIG. 14C is a cross sectional representation of an embodiment of a unit cell structure with light source 1402 and light source housing 1414, optical element 1450, and a light integration unit 1404 which is typically positioned on the plane of contact 1405 with a body surface. In this embodiment, the specific cross sectional geometry of the light integration unit is described by a height h, a variable lateral dimension b, a variable vertical dimension c, and a radius of curvature r describing the shape's edges at the plane of contact with a body surface. In one embodiment, the value of r is equal to h and the value of c and b are zero such that the form of the light integration element is hemispherical. In another embodiment, the values of b and c are equivalent and nonzero and therefore for a given value of r, the geometric form of the light integration element resembles a structure similar to a cube with the corners and edges nearer the plane of contact with the body surfaces taking on a rounded character. In yet another embodiment, the value of b and c are not equal yet are nonzero and therefore for given values of r, the geometric form of the light integration element resembles a structure similar to a rectangular prism with the corners and edges nearer the plane of contact with the body surface taking on a rounded character. For example, r may have a value ranging from 0.5 mm at a minimum to the value of h at a maximum. In another example, r can have a value ranging from 0.5 mm to 25 cm and is not constrained to a maximum of the value of h.

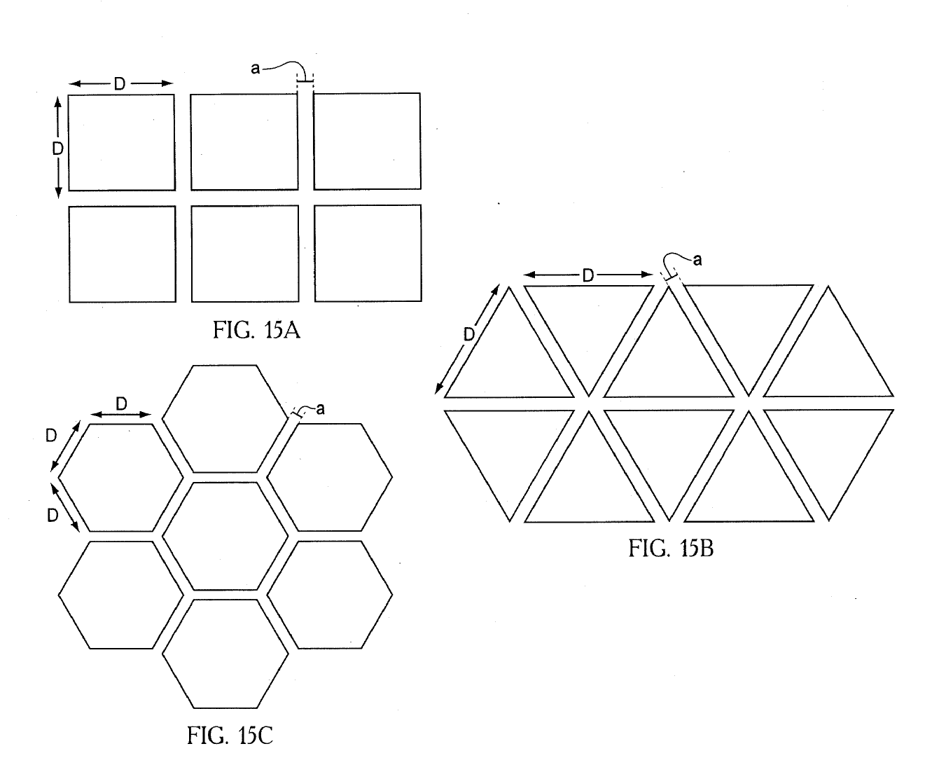

FIGS. 15A-C offers a schematic, plan view of several embodiments of particular individual unit cell geometry. FIG. 15A shows a representation of the aforementioned cuboid geometry, here depicted as a series of square shapes. FIGS. 15B-C further demonstrates respectively triangular and hexagonal shapes that can be employed. In all cases, each side of the unit cell is a length D with each unit cell being separated by a spacing a, and the placement of each shape is intended to efficiently incorporate these shapes such that the length D is considered large as compared to spacing a. Therefore the intention is to closely approximate a ‘close packed’ shape configuration, regardless of actual repeated shape used. For example, considering a cylindrical unit cell (not shown), the measurement D would alternatively describe the diameter of the unit cell.

In one embodiment, the selection of the size of each unit cell, represented here and earlier as length D, follows a specific design rule based upon the radius of curvature of a particular body surface that the light emitting element is applied to as well as the tolerance for deformation of a particular body surface. This situation is schematically represented by FIG. 16 where representative cross sectional unit cells 1603 are applied to a surface with a radius of curvature R. The goal of the device is to conform to a body surface using this unique unit cell approach to practically deliver uniformly distributed light. Therefore, maximum contact of the distal end of each unit cell with a particular body surface is desired and considered important in achieving this goal. Therefore, an allowable deformation by the unit cell structures of the typically relatively soft skin surface of a patient's body is referred to as distance d and is also included in this FIG. 15. An inset to FIG. 15 includes the geometric solution to find an expression for D in terms of the known quantities d and R. Symbolically,
D=2\sqrt{2dR−d²}.
These design criteria allows for customization of either individual or a range of light delivery devices with respect to particularly dissimilar body surfaces. Alternatively, the device is applied to a rigid body surface such that no value of deformation, d, is allowed. The distance d thus theoretically represents the maximum separation between the bottom plane of a unit cell structure and a body surface. In this case, in order to maintain a uniform light distribution delivered to a body surface, a range of values for D can be selected such that for a given radius of curvature, R, the value d remains small as compared to D. In practical application, D may be less than 5 cm.

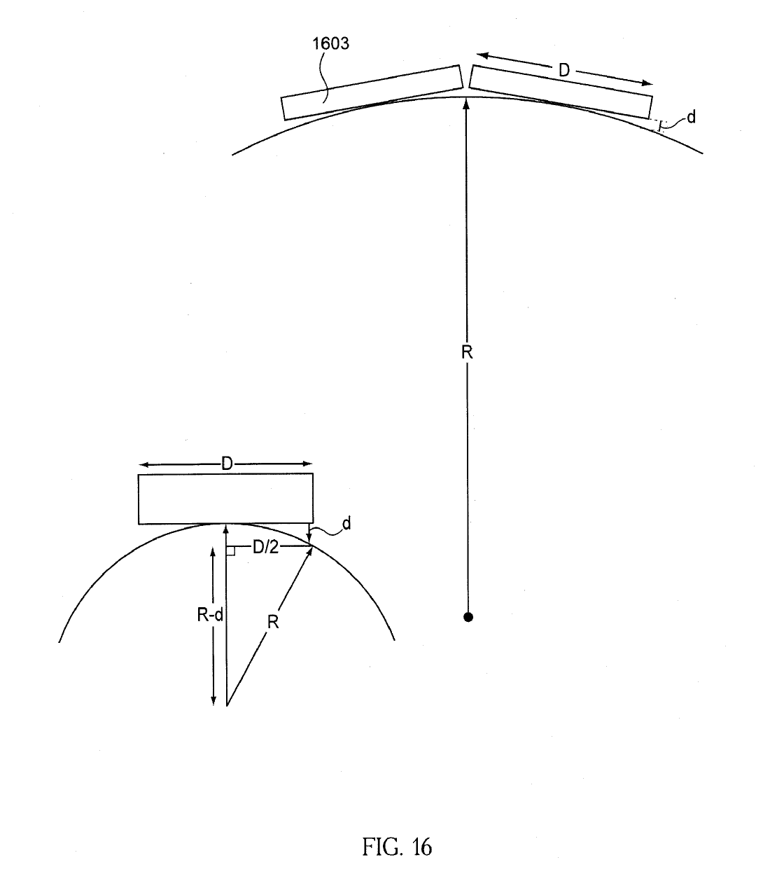

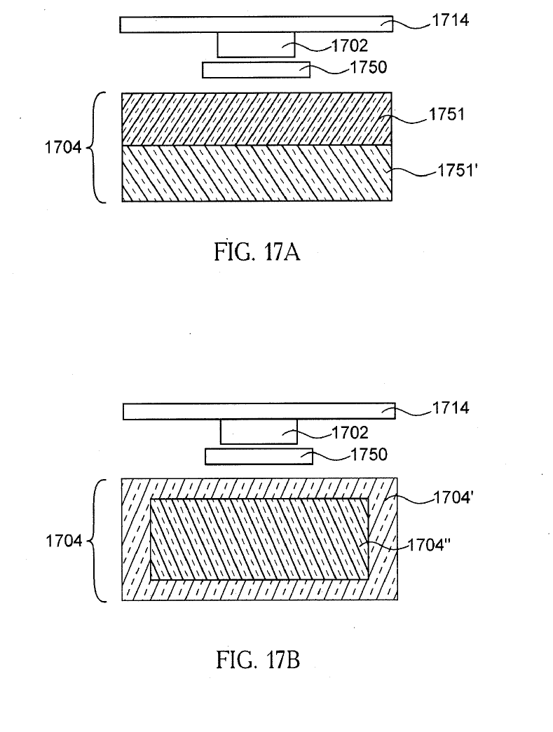

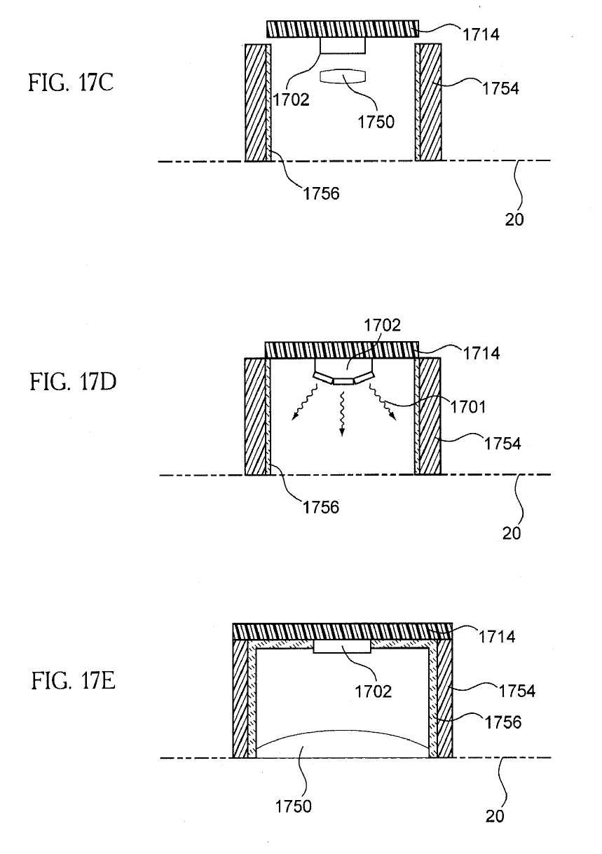

FIG. 17A is a cross sectional representation of an embodiment of a unit cell structure with light source 1702 and light source housing 1714, optical element 1750, and a light integration unit 1704. In this embodiment, the light integration structure is composed of two distinct materials, with a first one 1751 immediately disposed below the optical element and the second material 1751′ substantially located at the plane of contact with a body surface. One aspect of this embodiment is to create an index of refraction contrast by joining two dissimilar materials. The interface between two materials of dissimilar indices of refraction can present an opportunity to shape and otherwise condition light transmitting from one material to the other. Therefore, the distribution and therefore uniformity of the transmitted therapeutic light can be affected in a way beneficial to the therapeutic treatment. Various shapes and quantities of these two materials that differ from what is schematically depicted can be employed in keeping with the original description.

Further, an alternative configuration of the above concept is described. FIG. 17B is a cross sectional representation of an embodiment of a unit cell structure with light source/light source housing 1714, and optical element 1750 and a light integration unit 1704. In this embodiment, the light integration structure is composed of two distinct materials, with a first material 1704′ entirely surrounding the second material 1704″. The interface between two materials of dissimilar indices of refraction can present an opportunity to shape and otherwise condition light transmitting from one material to the other. Therefore, the distribution and therefore uniformity of the transmitted therapeutic light can be affected in a way beneficial to the therapeutic treatment. For example, the second material can be air. One aspect of this configuration can offer the advantage reduced transmission losses through the optical integrator medium while still. A second aspect of this configuration is that it can offer several material interfaces with which to shape and otherwise condition transmitted light. Various shapes and quantities of these two materials that differ from what is schematically depicted can be implemented in accordance with the above description.

FIG. 17C is a cross sectional representation of an embodiment of a unit cell structure with light source 1702 and light source housing 1714 and optical element 1750. Physical support is provided by a supporting wall structure 1754 which contacts the light source housing as well as the plane of contact with the body surface 20. This supporting wall structure may be coated with a material 1756 that is at least partially reflective. Alternatively, the optical properties of the supporting material are such that uniform light distribution is achieved by making use of physical phenomena including but not limited to total internal reflection. This supporting element may be made of metal, an elastomer, an acrylic, fused silica, a combination of any of the above or any other suitable material or combination of materials. As opposed to the above designs, this particular structure does not explicitly provide a solid material light integrator as part of the unit cell. However, the above structures can function in conjunction to provide a similar ability to distribute light uniformly to a contacted body surface. It an embodiment, it is intended that these support structures make contact with 15% or less of the body surface receiving the therapeutic treatment. Optical element 1750 functions to aid in the distribution of the light emitted from the light source 1702. This optical element 1750 may be a convex, concave, aspheric, diffractive, Fresnel type, or free form lens. It is also possible to incorporate this optical element 1750 directly into the light source housing 1714 feature. An alternative embodiment does not make use of a specific intermediary optical element.

FIG. 17D is a cross sectional representation of an embodiment of a unit cell structure with light sources 1702 and a light source housing 1714. Physical support is provided by a supporting wall structure 1754 which contacts the light source housing 1714 as well as the plane of contact with the body surface 20. This supporting wall structure may be coated with a material 1756 that is at least partially reflective. In this embodiment, one or more light sources 1702 are permanently positioned at one or more predetermined angles with respect to the plane of contact with the body surface 20 to emit light such that a substantially uniform distribution of light is transmitted to the body surface at the plane of contact with the cell support structure.

FIG. 17E is a cross sectional representation of an embodiment of a unit cell structure with light source 1702, light source housing 1714, and optical element 1750. Physical support is provided by a supporting wall structure 1754 which contacts the light source housing 1714 as well as the plane of contact with the body surface 20. This supporting wall structure 1754 may be coated with a material 1756 that is at least partially reflective. Additionally, select surfaces of the light source housing 1714 may also be coated with a material that is at least partially reflective. In this embodiment, the optical element 1750 is located at the distal end of the unit cell in close proximity to the plane of contact of the body surface. This optical element 1750 may be a converging, diverging, aspheric, diffractive, fresnel type, or free form lens, but may also be a filter, diffuser, or body which otherwise conditions the light.

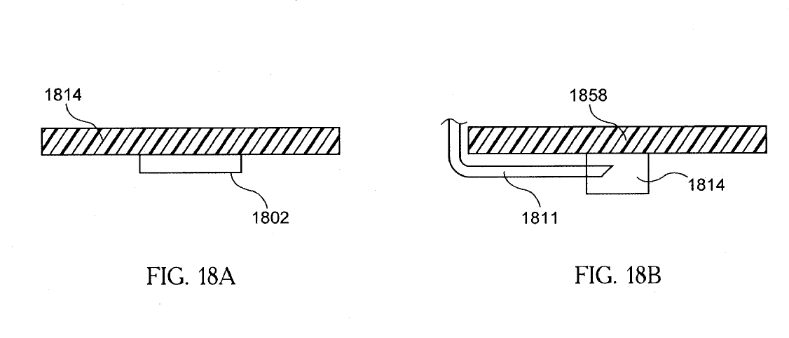

FIG. 18A depicts a light source 1802 and associated light source housing 1814 that has been described in the preceding text. This light source may be a semiconductor diode such as an LED or laser diode and its associated packaging. The housing serves to provide mechanical support to the light source. The light source is attached to the light source housing by any number of suitable mechanisms that may include solder, epoxy, ultrasonic bonding, thermal paste, or any combination thereof. The housing 1814 can also substantially comprise a printed circuit board to provide electrical connection to the semiconductor light source. As discussed previously, this light source housing can be mounted in or on a flexible substrate. This could include being molded-into the substrate or being separately attached in any other desired manner.

FIG. 18B depicts a light source and light source housing that has been described in the preceding text. In this embodiment, the light source is an optical fiber 1811 or group of optical fibers (not shown) which terminate in the vicinity of a specialized housing 1814. In this configuration, the fiber communicates light which is generated from a light source or group of light sources that are located separately. An advantage of this scheme is such that it can reduce the complexity of the device by effectively reducing the number of associated light sources. A support structure 1858 is provided for routing and aligning an optical fiber and for mechanical integrity. The optical fiber(s) 1811 as shown to be positioned horizontal to the body surface, but can be oriented in alternative ways as well. Not depicted are any number of optional optical elements that can be incorporated into the device to direct the light emitted from the terminating optical fiber. As will be appreciated by those skilled in the art, optical elements that direct light emitted from an optical fiber can be incorporated into the design of the device without departing from the scope of the invention. For example, a reflective surface such as a mirror can be positioned to direct emitted light in a substantially different dissimilar direction with respect to the orientation of the terminating fiber. The various schemes presented for an optical integrator also can be applied to this embodiment, where the optical fiber or fibers terminates into the optical integrator.

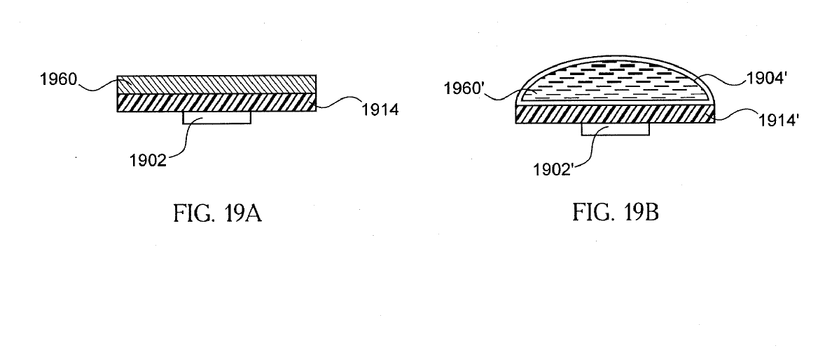

FIG. 19A depicts a variation on a light housing/light source element for a semiconductor diode light source. A heat sink element 1960 comprising a flat thermally conductive material is attached via solder, thermal paste, epoxy, ultrasonic bonding, or other compatible mechanism, to the light source housing and may alternatively be considered to be part of the light source housing 1914. In this embodiment, there exists a direct thermal path from the semiconductor diode light source 1902 to this heat sink element 1960. As will be appreciated by those skilled in the art, the heat sink 1960 can be exposed to air to maximize its exposure for convective heat transfer and thus enhanced thermal dissipation. The heat sink 1960 may be comprised of a metal, a ceramic, a polymer, an inorganic material such as graphite, or any combination thereof.

FIG. 19B depicts an alternative embodiment of a light housing 1914′ for a semiconductor diode light source 1902′. A heat sink reservoir 1960′ may be enclosed by a support structure 1904′. In this embodiment, there exists a direct thermal path from the semiconductor diode light source 1902′ to this heat sink reservoir 1960′. The heat sink reservoir 1960 is considered to have sufficient heat capacity to absorb the heat generated during the operation of a semiconductor diode light source 1902′ during an application of phototherapy. As is depicted, heat sink reservoir 1960′ can consist of a conventional heat absorbing material with significant heat capacity such as water. Alternatively, this material can be a phase change material such as a salt hydride or paraffin. The essential property of such a material is that it typically can absorb a relatively significant amount of heat, for example at a temperature equivalent to its melting point, before releasing the energy in the form of a phase change. Preferably, this phase change would occur at or around body temperature of approximately 37 C. Alternatively, the heat sink reservoir 1960′ is composed of a metal such as copper or aluminum.

An applied concept for the described device in all of its embodiments is the use of this device alongside a targeting method. Typically, areas of a patients body surface in need of phototherapy are of varying shape and size; therefore, rather than custom manufacture light delivery units to conform to these sizes, an intermediate separate object can be arranged to only expose desired areas to the treatment light.

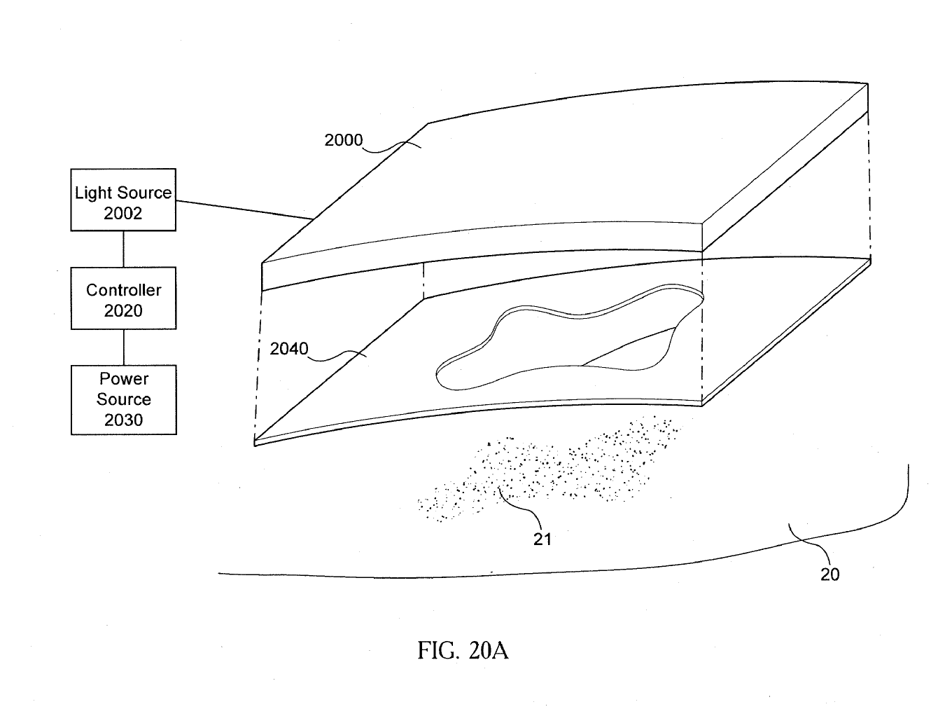

FIG. 20A shows an exploded view of the basic components of one embodiment of this concept. An affected area 21 on a patient's body surface 20 is of an arbitrary shape. A separate targeting mask 2040 may be a continuous light absorbing material with a predetermined ‘window’ area that is reasonably identical to the shape and size of the prescribed area on the patient's body surface. This mask can be have an adhesive coating on one or more sides to adhere to the patient body surface, the light delivery device, or both. Other mechanisms are possible to attach the mask to the target surface including, but not limited to, straps or an elastic grip. Alternatively, the mask can be attached directly to the therapeutic device or can be considered part of the therapeutic device. This mask can for example be constructed from a flexible material such as cloth, an elastomer, foam, a non-woven synthetic, or other suitable material and is 0.1 to 5 mm in thickness, but preferably 0.5 to 2 mm in thickness. A light delivery device 2000 that is flexible and conformal to a patient body surface 20 and is attached to a light source 2002, controller 2020, and power source 2030 is depicted and intended to be used in concert with the targeting mask.

FIG. 20B illustrates a collapsed, ‘in service’ view of the targeting element 2040 in between the delivery device 2000 and the patient body surface 20. In this depiction, the mask element is in intimate contact with the light delivery device and the patient body surface. It is important to note that the lateral dimensions of the targeting mask can be larger than the light delivery device to ensure that therapeutic light does not reach the patient body surface beyond the mask borders. In an embodiment, the mask can extend far beyond the periphery of the light delivery device to an extent that it can be contiguous over a certain part of an extremity. For example, the mask could wrap entirely around an arm and be secured in a similar fashion to that of a typical wristband or elbow brace. In an alternative embodiment, the mask material can be made up of a gel, liquid, aqueous or oil based suspension, or other physically or chemically blocking material that would at least partially absorb incident therapeutic light. In this embodiment, the mask material would selectively be applied to areas of a body surface which are desired to either receive either a zero amount or alternatively a substantially reduced amount of therapeutic light as compared to a desired treatment location such as an affected area. The light delivery device 2000 is attached to a light source 2002, controller 2020, and power source 2030 is also depicted.

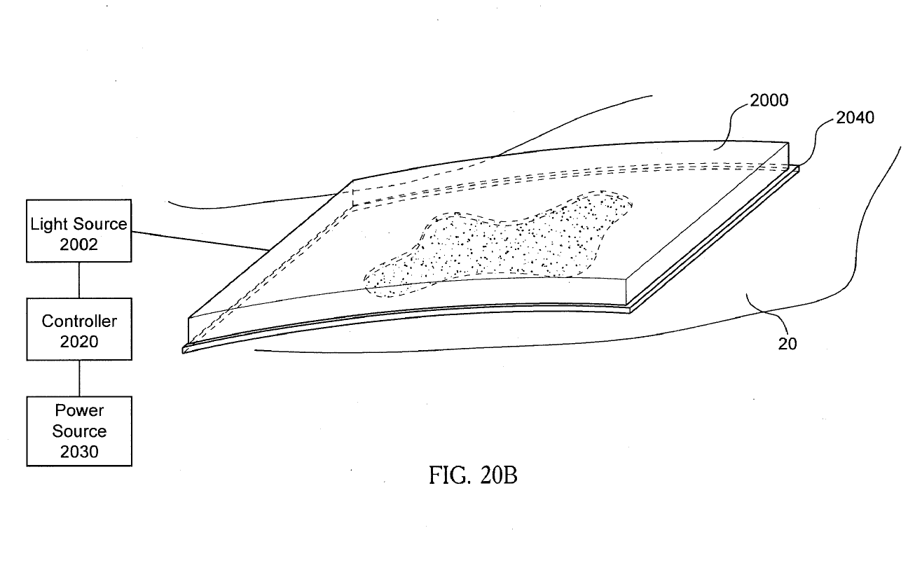

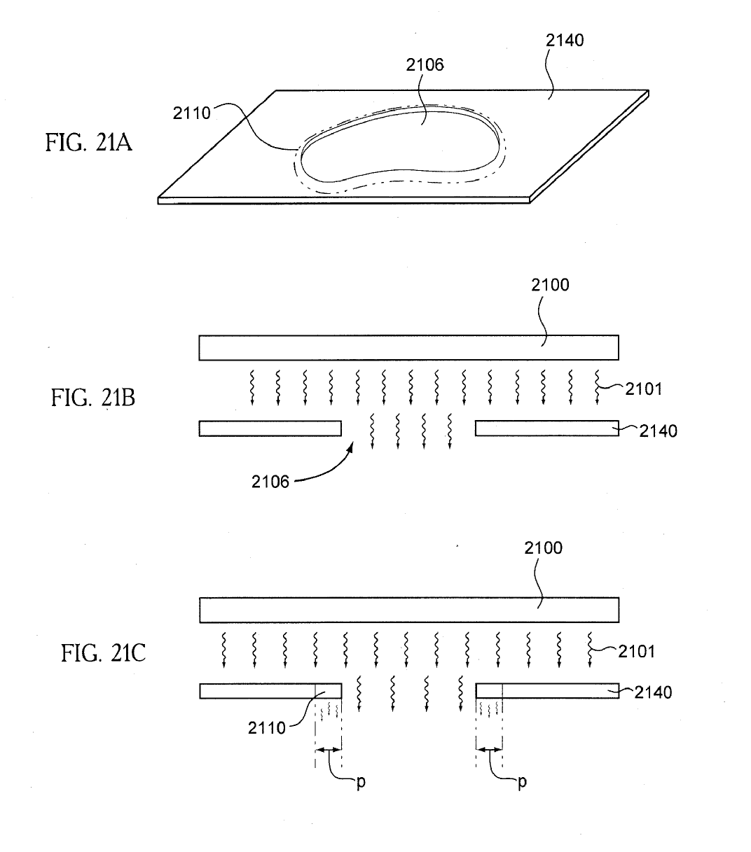

FIG. 21A depicts the features of an embodiment of a masking element 2140. Chief among these features is a well defined light transmissive region 2106 that is at least partially transmissive to light. The mask may include a border region 2110 which is the area surrounding this transmissive region, of a lateral dimension p into its exterior periphery. This border region 2110 may extend for a distance of 0.0 to 10 mm but more preferably 2-5 mm. The remaining portion of the masking element can be composed of a material which is absorbing to the light emitted by the associated phototherapy device.

FIG. 21B is a cross sectional schematic view of a light delivery device 2100 emitting treatment light 2101 with a representative masking element 2140 between this device and a body surface (not shown). The light transmissive region 2106 may have no physical material presence thus theoretically allowing 100% transmission or may be a distinct material which is at least partially transmissive to the therapeutic light. In the latter case, this material may be a silicone elastomer, an acrylic, polycarbonate, or other suitably light transmissive material.

FIG. 21C is cross sectional schematic view of a light delivery device 2100 emitting treatment light 2101 with a representative masking element 2140 between this device and a body surface. Feature 2110 is a border region which has distinctly reduced transmission, such as a range of 10%-90% or alternatively, 30%-60%, of that of the specifically defined light transmission region 2106. This feature is to reduce the overall phototherapy dose delivered in and around the borders of the affected area to be treated.

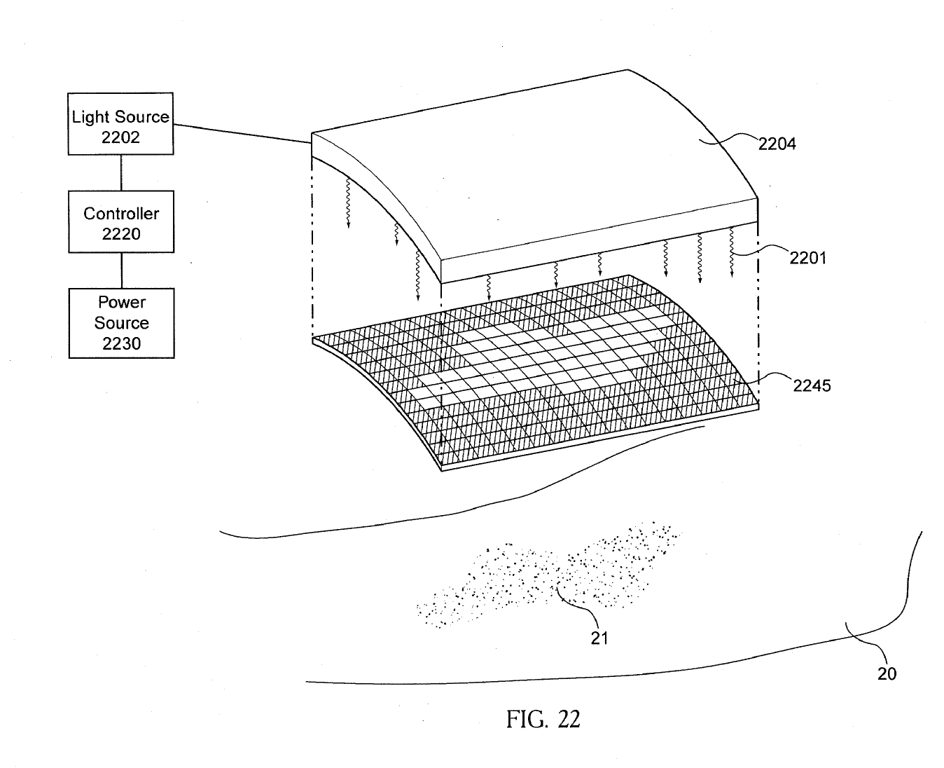

An alternative embodiment of an applied concept for delivering targeted phototherapy is depicted in FIG. 22. This embodiment consists of a flexible and conformal light delivery element 2204 that is emitting therapeutic light 2201, and a light source 2202 that is connected to a controller 2120 and a power supply 2130. The controller 2120 is also connected to a flexible and conformal masking element 2245 that is essentially an externally programmable membrane. This membrane functions similar to that of a liquid crystal display that is able to, in a pixilated and patterned fashion, selectively at least partially block or transmit light emitted from the light delivery device. In an embodiment, as is depicted, the pattern of such selectivity closely matches that of the affected area 21 on a patient body surface 20 that requires phototherapy. This registry can be programmed in advance or can be generated by active sensors in this mask element after placement on the surface to be treated but prior to actual phototherapy application.

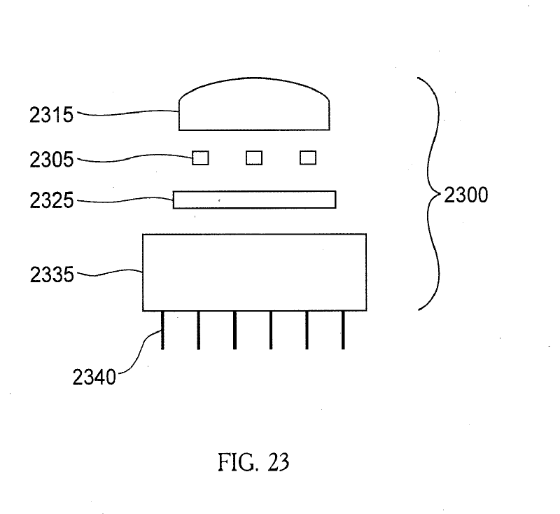

In one embodiment, the radiation devices are light emitting diodes (LEDs) and the material between the LEDs and the covering which interfaces with the body surface is transparent to the light emitted from the LEDs. In one embodiment, the LEDs emit ultraviolet light in the wavelength range 200-400 nm. In another embodiment, the LEDs emit visible light in the wavelength range of 400-800 nm. In another embodiment, the LEDs emit infrared light in the wavelength range of 800-2000 nm. The LEDs are chips which are then assembled into modules, or light sources, which can be manipulated into a larger device. FIG. 23 depicts a radiation source 2300 which consists of LED chips 2305, a chip covering or encapsulant 2315, a chip submount 2325 and a base platform 2335.

The base platform 2335 can be produced from a substance with a thermal conductivity to efficiently conduct heat that may be generated by the LEDs during operation away from the LED devices and, by association, a patient's body surface. The base can be microfabricated, molded, machined, or otherwise produced by techniques well known to those skilled in the art. The base can further be shaped to conduct heat in an optimal manner. For example, fins 2340 can be fabricated, deposited, or mechanically or otherwise attached onto the base platform. In another embodiment, a thermoelectric cooler is attached to the base platform. The base can further be processed such that it may become a component in a circuit on the irradiating device 100. In an embodiment, the substrate of the radiative device 100 is made so that the base (and module) can easily press-fit into the radiating device. The portable irradiating device then has contacts thereon which provide for electrical communication between the controller and the module 2300.

Covering 2315 is made from a material transparent to the radiation emitted from the device. In the case where the chips 2305 emit ultraviolet radiation, the covering 2315 can be produced from a material such as silicone, fluorinated-ethylene propylene (FEP), fused silica, or other suitably light transmissive material. It is preferable that the covering 2315 be of a similar index of refraction as compared to that of the semiconductor chip (as described in Example 3, above), so as to minimize reflection at the interface of the two materials. Covering 2315 can further contain additional interfaces which serve to condition the light as it is emitted from the semiconductor material. In an embodiment, the LED is an ultraviolet LED which emits light from a surface with dimension of about 1 square mm or smaller. The covering conditions the light so that the light is distributed over an area of at least 1 cm² from the mount 2325. In another embodiment, the covering conditions the light so that the light is distributed over an area of between 0.4 cm² and 1 cm² from the smaller mount. In another embodiment, the covering conditions the light such that the light is distributed to an area less than 4 cm². In yet another embodiment, the covering conditions the light to spread over an area greater than 1 cm². The conditioned light may be distributed in a uniform fashion or may be distributed in a desired pattern. When 1-2 cm² (for example) is used, the covering 2315 can diffuse light from a mount less than about 1-3 mm² to a region 1-2 cm² over a distance of between 0.5 and about 5 mm (the distance between the LED devices and the skin).

### EXAMPLE 1

A ray tracing calculation was performed to show the effect of a light integrator on the uniformity of the output power at the exit plane of the device.

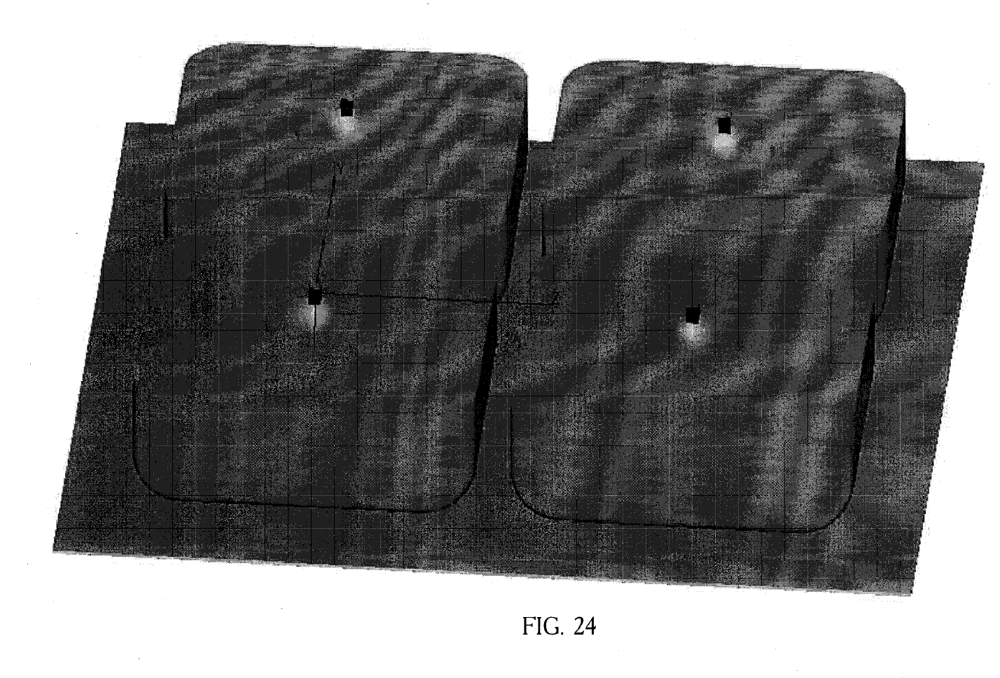

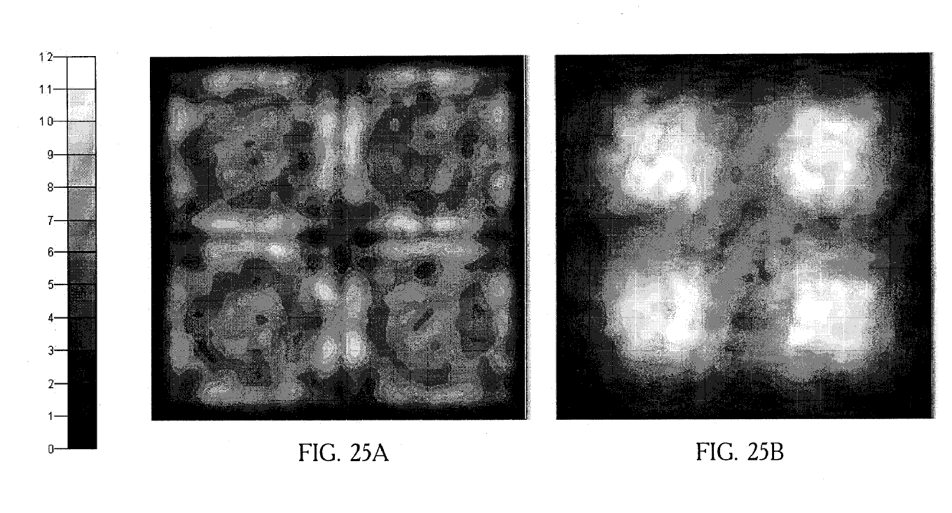

The resulting output of four LED emitters to a flat body surface was simulated. The four LEDs are positioned on a square grid with an 11.5 mm spacing between the centers of each LED. The distance to the body surface is 5.5 mm. The integrators consist of silicone rubber geometrical shapes that are approximately cuboid in structure with rounded edges and corners. For simulation purposes, the refractive index of such transparent structures was set to 1.5. The size of the integrators is 10×10×4 millimeters, and the edges are rounded with a radius of 1 mm. There is a negative lens incorporated into the top side of the integrator element which faces towards each respective LED. The radius of this lens is 1 mm. A schematic diagram from an approximately ¾ viewpoint is depicted in FIG. 24. The LED's are assumed to be Lambertian emitters with a surface area of 0.35 mm×0.35 mm. The ray tracing software (TracePro, Lambda Research Corporation) simulated two distinct models. The resultant simulated optical intensity distribution profile at the exit plane of the integrator element is depicted in FIG. 25A. A second case without the integrator structures present, though otherwise identical, is depicted in FIG. 25B. A relative intensity scale is also included. The use of light integrator elements, as provided for in FIG. 25A, improves the light distribution intensity profile. Additionally, as will be appreciated by those skilled in the art, optimization of the shape of the light integrators can be modified to further achieve an even flatter distribution profile for the device.

A variety of kits are also contemplated for use with this invention. For example, patients could be provided with kits that have a plurality of radiation applicators with different sizes and shapes and in which each size and shape can be fit together. The applicators could be configured to provide the same radiation for the same amount of time, or could be applicators having different radiation types and/or amounts and/or time configurations. The applicators can be fit together and then further adapted to communicate with a computer program to customize the type, quality, quantity and/or location of treatment to a pre-defined region. For example, where it would be desirable to provide a first quality of treatment at a first time and a second quality of treatment at a second time, or where it is anticipated that the amount of radiation and/or time of radiation required would change during the course of delivering the therapy. Thus, for example, a first radiation applicator having the ability to deliver a first amount of radiation at a first amount of time, could be provided with a second radiation applicator having the ability to deliver a second amount of radiation for a second amount of time. Thus enabling a kit to be provided that has the ability to slowly increase therapy over time, increase and then decrease therapy over time, or decrease therapy over time.

As will be appreciated by those skilled in the art, a variety of methods can be employed to treat a prescribed area of a target body surface with phototherapy. In one such embodiment, a prescribed area is treated by: a) applying a light therapy device adapted to conform to the target body surface; (b) selectively delivering a therapeutic dose of light to at least a portion of the target body surface. This method can used for a variety of dermatological treatments including, but not limited to, the following: psoriasis, vitiligo, atopic dermatitis, infection, sun tanning, acne, skin cancer, actinic keratosis, hair removal, dermal vascular lesions and pigmentation, skin rejuvenation, and bilirubin. Another embodiment is the use of this phototherapy with a photosensitizer, where the treatment method includes: (a) administering a photosensitizer to the patient; (b) applying a light therapy device adapted to conform to the target body surface; (c) delivering a therapeutic dose of light to at least a portion of the target body surface.

In another embodiment, a prescribed area is treated by: a) applying a light therapy device adapted to conform to the target body surface and comprising a plurality of light sources; (b) using a detector to determine the presence of target tissue; (c) activating one or more of the light sources to the target tissue to deliver a therapeutic dose of light. In this embodiment, the detector detects one or more of the following skin characteristics: temperature, electrical impedance, photoreflectance, thickness, hardness, moisture, and acoustic reflections. In this embodiment, photoreflectance measures one of roughness, color, or fluorescence.

In yet another embodiment, a prescribed area is treated by: a) applying a targeting mask to the target body surface; (b) applying a light therapy device adapted to conform to the target body surface and at least partially coupled to the targeting mask; (c) delivering a therapeutic dose of light to at least a portion of the target body surface through the targeting mask. In yet another embodiment, a prescribed area is treated by: a) applying a substance to a non-prescribed region of the body surface which at least partially blocks therapeutic light; (b) applying a light therapy device to the prescribed region and at least partially to the non-prescribed region, the device being adapted to conform to the target body surface; (c) delivering a therapeutic dose of light to at least a portion of the prescribed region. In this embodiment, the light blocking substance is one of a cream, lotion, gel, ointment, paste or fluid.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A therapeutic treatment apparatus adapted and configured to conform to a target region of a patient, the apparatus comprising:
a plurality of light sources adapted and configured to couple to a flexible substrate to deliver light to the target region,
a power supply coupled to the light sources and operable to provide power to the light sources, and
a controller coupled to the light sources and the power supply and operable to control the operation of the light sources,
wherein the therapeutic treatment apparatus is disposed adjacent a light integrator in at least a portion of an optical path for the light between the light sources and the target region of the patient during deployment.

2. The therapeutic treatment apparatus of claim 1 wherein each light source further comprises one or more light emitting diodes.

3. The therapeutic treatment apparatus of claim 1 wherein each light source further comprises one or more laser diodes.

4. The therapeutic treatment apparatus of claim 2 or 3 wherein the diode is positioned relative to a surface of the flexible substrate to deliver light at one or more prescribed angles with respect to the target region of the patient's body surface.

5. The therapeutic treatment apparatus of claim 1 wherein at least one light sources emits light between wavelength range of 200-2,000 nm.

6. The therapeutic treatment apparatus of claim 1 wherein the flexible substrate is a substrate consisting of rubber, cloth; thermoplastic elastomer, thermoplastic, fabric, or flexible metal.

7. The therapeutic treatment apparatus of claim 1 further comprising a single-use layer positioned between light delivered by the light sources and the target region of the patient's body surface.

8. The therapeutic treatment apparatus of claim 1 wherein the light integrator is formed from a rigid or semi-rigid material further adapted and configured to at least partially transmit light.

9. The therapeutic treatment apparatus of claim 8 wherein the light integrator is adapted and configured to internally reflect the light to substantially uniformly distribute the light onto the target region of the patient's body surface.

10. The therapeutic treatment apparatus of claim 8 wherein the light integrator is adapted and configured to use a total internal reflection to distribute the light onto the target region of the patient's body surface.

11. The therapeutic treatment apparatus of claim 10 wherein the internal reflection is substantially uniform.

12. The therapeutic treatment apparatus of claim 8 wherein one or more lower edges of the light integrator are adapted and configured to have a minimum radius of curvature of 0.5 mm and maximum radius of curvature of 25 cm.

13. The therapeutic treatment apparatus of claim 8 wherein the light integrator further comprises silicone rubber.

14. The therapeutic treatment apparatus of claim 1 wherein the light integrator is at least partially further comprised of a support structure adapted and configured to separate the light sources and the target region of the patient's body surface.

15. The therapeutic treatment apparatus of claim 14 wherein the support structure further comprises a partially reflective support structure.

16. The therapeutic treatment apparatus of claim 14 wherein the support structure is adapted and configured to contact <15% of the target region of the patient's body surface.

17. The therapeutic treatment apparatus of claim 14 wherein the light integrator further comprises a lens adapted and configured to be positioned between the light source and the target region of the patient's body surface.

18. The therapeutic treatment apparatus of claim 1 wherein the substrate further comprises a substrate at least partially transmissive to light.

19. The therapeutic treatment apparatus of claim 18 wherein the substrate is silicone rubber.

20. The therapeutic treatment apparatus of claim 1 wherein the controller is configurable to selectively control one or more treatment parameters.

21. The therapeutic treatment apparatus of claim 1 wherein the controller is configurable to selectively provide one or more patient specific codes.

22. The therapeutic treatment apparatus of claim 1 wherein the controller is configurable to selectively control one or more treatment parameters for a specific target region of patient.

23. The therapeutic treatment apparatus of claim 1 wherein apparatus further comprises sensors in communication with the controller and configured to detect proper placement of the apparatus on patient.

24. The therapeutic treatment apparatus of claim 20 wherein treatment parameters are selected from the group consisting of: duration of treatment, treatment frequency, or total numbers of available treatments.

25. The therapeutic treatment apparatus of claim 1 wherein the apparatus further comprises an attachment mechanism adapted and configured to attach the apparatus to the patient.

26. The therapeutic treatment apparatus of claim 25 wherein the attachment mechanism is selected from the group consisting of: adhesive, straps, material wraps, or a cuff.

27. The therapeutic treatment apparatus of claim 1 wherein the apparatus further comprises a heat collector adapted and configured to absorb heat generated by the light sources.

28. The therapeutic treatment apparatus of claim 27 wherein the heat collector further comprises a material integrated with each light source wherein the material is selected from the group consisting of a heat conductive material or a heat absorbing material.

29. The therapeutic treatment apparatus of claim 1 wherein the apparatus further comprises a targeting mask adapted and configured to at least partially block therapeutic light from a first region of a patient and at least partially transmit therapeutic light to a second region of a patient.

30. The therapeutic treatment apparatus of claim 29 wherein the targeting mask further comprises an attachment mechanism adapted and configured to attach the apparatus to the patient.

31. The therapeutic treatment apparatus of claim 30 wherein the attachment mechanism further comprises adhesive.

32. The therapeutic treatment apparatus of claim 29 wherein the mask further comprises at least one flexible material.

33. The therapeutic treatment apparatus of claim 32 wherein the flexible material is selected from the group consisting of foam, rubber, plastic, synthetic fabric, natural fabric, or elastomer.

34. A therapeutic treatment apparatus adapted and configured to contact a target surface of a patient comprising:
a light source,
a power supply coupled to the light source and operable to provide power to the light source,
a power switch coupled to the light source and the power supply and operable to control delivery of power from the power supply to the light source, and
a light integrator adapted and configured to selectively transmit light from the light source to a target surface.

35. A therapeutic treatment apparatus adapted and configured to conform to a surface of a patient comprising:
a plurality of light sources flexibly interconnected to at least one other light source,
a power supply coupled to the light sources and operable to provide power to the light sources,
a controller coupled to the light sources and the power supply and operable to control the operation of the light sources,
wherein each light source further comprises an optical waveguide adapted and configured to selectively distribute light onto the target surface.

36. The therapeutic treatment apparatus of claim 35 wherein the waveguide further comprises silicone rubber.

37. The therapeutic treatment apparatus of claim 35 wherein the waveguide further comprises optical fibers.

38. A therapeutic treatment apparatus adapted and configured to conform to a patient comprising:
a plurality of light sources adapted and configured to deliver light wherein the light sources are coupled to an elastomeric substrate and further wherein the substrate is comprised of a material having a durometer of less than or equal to shore 70 A and is at least partially transmissive to the light,
a power supply coupled to the light sources and operable to provide power to the light sources, and
a controller coupled to the light sources and the power supply wherein the controller is operable to control the operation of the light sources.

39. A therapeutic treatment apparatus adapted and configured to conform to a target surface of a patient comprising:
a plurality of light sources,
a power supply coupled to the light sources and operable to provide power to the light sources,
a controller coupled to the light sources and the power supply and operable to control the operation of the light sources,
wherein the light sources are flexibly connected and further wherein the distance between at least two of the light sources is less than or equal to the distance between light sources and the target surface.

40. A therapeutic treatment apparatus system comprising:
a light source,
a controller coupled to the light source,
a power supply coupled to the light source and the controller and operable to provide power to the system,
a fiber optic fiber adapted and configured to deliver light from the light source to a flexible substrate adapted and configured to conform to a patient's body surface,
wherein the fiber optic fibers terminate into a light integrator which substantially uniformly distributes light onto target surface.

41. A therapeutic treatment apparatus adapted and configured to conform to a target region of a patient comprising:
a plurality of light sources coupled to a flexible substrate,
a power supply coupled to the light sources and operable to provide power to the light sources,
a controller coupled to the light sources and the power supply and operable to control the operation of the light sources, and
a light integrator adapted and configured to be positioned in at least a portion of an optical pathway between the light source and the target region of the patient,
wherein the light sources are spaced such that D=2\sqrt{2dR−d²} where D is a width of light integrator, R is a radius of curvature of the target region, and d is a sum of tissue compression and an optically allowable gap between the light integrator and a target region.

42. A therapeutic treatment apparatus adapted and configured to conform to a patient's body comprising:
a plurality of light sources,
a power supply coupled to the light sources and operable to provide power to the light sources, and
a controller coupled to the light sources and the power supply and operable to control the operation of the light sources,
wherein the light sources are adapted and configured to illuminate such that the light exiting the light source is substantially parallel with the body.

43. A method of treating a prescribed area of a target body surface comprising the steps of:
(a) applying a light therapy device adapted to conform to the target body surface; and
(b) selectively delivering a therapeutic dose of light to at least a portion of the target body surface.

44. The method of claim 43 where the method provides treatment for a clinical indication selected from the group consisting of:
(a) psoriasis
(b) vitiligo
(c) atopic dermatitis
(d) infection
(e) sun tanning
(f) acne
(g) skin cancer
(h) actinic ketatosis
(i) hair removal
(j) dermal vascular lesions or pigmentation
(k) skin rejuvenation
(l) bilirubin

45. The method of claim 43 further comprising chilling a device prior to applying light therapy device to a body surface.

46. A method of treating a prescribed area of a target body surface comprising the steps of:
(a) administering a photosensitizer to a patient;
(b) applying a light therapy device adapted and configured to conform to the target body surface; and
(c) delivering a therapeutic dose of light to at least a portion of the target body surface.

47. A method of treating a prescribed area of a target body surface comprising the steps of:
(a) applying a light therapy device adapted to conform to the target body surface and comprising a plurality of light sources;
(b) using a detector to determine at least one property of target tissue; and
(c) selectively activating one or more of the light sources in response to the detector to deliver a therapeutic dose of light to the target tissue.

48. The method of claim 47 further comprising the step of detecting one or more of the following properties: temperature, electrical impedance, photoreflectance, thickness, hardness, moisture, acoustic reflections.

49. The method of claim 48 wherein the step of measuring photo reflectance includes the step of measuring one or more of: roughness, color, or fluorescence.

50. A method of treating a prescribed area of a target body surface comprising the steps of:
(a) applying a targeting mask to the target body surface;
(b) applying a light therapy device adapted and configured to conform to the target body surface and at least partially coupled to the targeting mask; and
(c) delivering a therapeutic dose of light to at least a portion of the target body surface through the targeting mask.

51. A method of treating a prescribed area of a target body surface comprising the steps of:
(a) applying a substance to a non-prescribed region of a body surface which at least partially blocks therapeutic light;
(b) applying a light therapy device adapted and configured to conform to the target body surface to a prescribed region of the body surface and at least partially to the non-prescribed region;
(c) delivering a therapeutic dose of light to at least a portion of the prescribed region.

52. The method of claim 51 where the light blocking substance is one of a cream, lotion, gel, ointment, paste, or fluid.
